(19) United States
(12) Patent Application Publication
(10) Pub. No.: US 2003/0040000 A1
(43) Pub. Date: Feb. 27, 2003

(54) Methods for attaching nucleic acid molecules to electrically conductive surfaces

(76) Inventors: Dennis M. Connolly, Rochester, NY (US); Charles D. DeBoer, Palmyra, NY (US); David R. Chafin, Rochester, NY (US); Richard S. Murante, Rochester, NY (US)

Correspondence Address: Michael L. Goldman, NIXON PEABODY LLP, Clinton Square, P.O. Box 31051, Rochester, NY, 14603-1051, US

(21) Appl. No.: 10/159,429

(22) Filed: May 30, 2002

Related U.S. Application Data
(60) Provisional application No. 60/310,937, filed on Aug. 8, 2001.

(51) Int. Cl. C12Q 1/68; C12Q 1/00; B05D 3/00

(52) U.S. Cl. 435/6; 427/2.11; 205/777.5

(57) ABSTRACT

The present invention relates to a method of attaching nucleic acid molecules to two different electrical conductors, where a first set of oligonucleotide probes is attached to the first electrical conductors with an attachment chemistry which binds the first set of oligonucleotide probes to the first electrical conductors but not to the second electrical conductors. Then, a second set of oligonucleotide probes is attached to the second electrical conductors. The present invention also provides methods for attaching nucleic acid molecules to electrical conductors using a masking agent and methods for attaching nucleic acid molecules to electrical conductors by electrostatic attraction so that the oligonucleotide probes are chemically bound to the electrical conductors. The present invention also discloses methods and devices for detecting a target nucleic acid molecule in a sample.

This application claims the benefit of U.S. Provisional Patent Application Serial No. 60/310,937, filed Aug. 8, 2001, which is hereby incorporated by reference in its entirety.

### FIELD OF THE INVENTION

The present invention relates to devices and methods for the collection, purification and genetic characterization of nucleic acids, such as deoxyribonucleic acids (DNA) or ribonucleic acids (RNA), from fluid samples.

### BACKGROUND OF THE INVENTION

Nucleic acids, such as DNA or RNA, have become of increasing interest as analytes for clinical or forensic uses. Powerful new molecular biology technologies enable one to detect congenital or infectious diseases. These same technologies can characterize DNA for use in settling factual issues in legal proceedings, such as paternity suits and criminal prosecutions.

For the analysis and testing of nucleic acid molecules, amplification of a small amount of nucleic acid molecules, isolation of the amplified nucleic acid fragments, and other procedures are necessary. The science of amplifying small amounts of DNA have progressed rapidly and several methods now exist. These include linked linear amplification, ligation-based amplification, transcription-based amplification and linear isothermal amplification. Linked linear amplification is described in detail in U.S. Pat. No. 6,027,923 to Wallace et al. Ligation-based amplification includes the ligation amplification reaction (LAR) described in detail in Wu et al., Genomics, 4:560 (1989) and the ligase chain reaction described in European Patent No. 0320308B1. Transcription-based amplification methods are described in detail in U.S. Pat. Nos. 5,766,849 and 5,654,142, Kwoh et al., Proc. Natl. Acad. Sci. U.S.A., 86:1173 (1989), and PCT Publication No. WO 88/10315 to Ginergeras et al. The more recent method of linear isothermal amplification is described in U.S. Pat. No. 6,251,639 to Kurn.

The most common method of amplifying DNA is by the polymerase chain reaction (“PCR”), described in detail by Mullis et al., Cold Spring Harbor Quant. Biol., 51:263-273 (1986), European Patent No. 201,184 to Mullis, U.S. Pat. No. 4,582,788 to Mullis et al., European Patent Nos. 50,424, 84,796, 258017, and 237362 to Erlich et al., and U.S. Pat. No. 4,683,194 to Saiki et al. The PCR reaction is based on multiple cycles of hybridization and nucleic acid synthesis and denaturation in which an extremely small number of nucleic acid molecules or fragments can be multiplied by several orders of magnitude to provide detectable amounts of material. One of ordinary skill in the art knows that the effectiveness and reproducibility of PCR amplification is dependent, in part, on the purity and amount of the DNA template. Certain molecules present in biological sources of nucleic acids are known to stop or inhibit PCR amplification (Belec et al., Muscle and Nerve, 21(8):1064 (1998); Wiedbrauk et al., Journal of Clinical Microbiology, 33(10):2643-6 (1995); Deneer and Knight, Clinical Chemistry, 40(1):171-2 (1994)). For example, in whole blood, hemoglobin, lactoferrin, and immunoglobulin G are known to interfere with several DNA polymerases used to perform PCR reactions (Al-Soud and Radstrom, Journal of Clinical Microbiology, 39(2):485-493 (2001); Al-Soud et al., Journal of Clinical Microbiology, 38(1):345-50 (2000)). These inhibitory effects can be more or less overcome by the addition of certain protein agents, but these agents must be added in addition to the multiple components already used to perform the PCR. Thus, the removal or inactivation of such inhibitors is an important factor in amplifying DNA from select samples.

On the other hand, isolation and detection of particular nucleic acid molecules in a mixture requires a nucleic acid sequencer and fragment analyzer, in which gel electrophoresis and fluorescence detection are combined. Unfortunately, electrophoresis becomes very labor-intensive as the number of samples or test items increases.

For this reason, a simpler method of analysis using DNA oligonucleotide probes is becoming popular. New technology, called VLSIPS™, has enabled the production of chips smaller than a thumbnail where each chip contains hundreds of thousands or more different molecular probes. These techniques are described in U.S. Pat. No. 5,143,854 to Pirrung et al., PCT Publication No. WO 92/10092, and PCT WO 90/15070. These biological chips have molecular probes arranged in arrays where each probe ensemble is assigned a specific location. These molecular array chips have been produced in which each probe location has a center to center distance measured on the micron scale. Use of these array type chips has the advantage that only a small amount of sample is required, and a diverse number of probe sequences can be used simultaneously. Array chips have been useful in a number of different types of scientific applications, including measuring gene expression levels, identification of single nucleotide polymorphisms, and molecular diagnostics and sequencing as described in U.S. Pat. No. 5,143,854 to Pirrung et al.

Array chips where the probes are nucleic acid molecules have been increasingly useful for detection for the presence of specific DNA sequences. Most technologies related to array chips involve the coupling of a probe of known sequence to a substrate that can either be structural or conductive in nature. Structural types of array chips usually involve providing a platform where probe molecules can be constructed base by base or covalently binding a completed molecule. Typical array chips involve amplification of the target nucleic acid followed by detection with a fluorescent label to determine whether target nucleic acid molecules hybridize with any of the oligonucleotide probes on the chip. After exposing the array to a sample containing target nucleic acid molecules under selected test conditions, scanning devices can examine each location in the array and quantitate the amount of hybridized material at that location. Alternatively, conductive types of array chips contain probe sequences linked to conductive materials such as metals. Hybridization of a target nucleic acid typically elicits an electrical signal that is carried to the conductive electrode and then analyzed.

Techniques for forming sequences on a substrate are known. For example, the sequences may be formed according to the techniques disclosed in U.S. Pat. No. 5,143,854 to Pirrung et al., PCT Publication No. WO 92/10092, or U.S. Pat. No. 5,571,639 to Hubbell et al. Although there are several references on the attachment of biologically useful molecules to electrically insulating surfaces such as glass (http://www.piercenet.com/Technical/default.cfm?tmp1=../Lib/ViewDoc.cfm&doc=3483; McGovern et al., Langmuir, 10:3607-3614 (1994)) or silicon oxide (Examples 4-6 of U.S. Pat. No. 6,159,695 to McGovern et al.), there are few examples of effective molecular attachment to electrically conducting surfaces except for gold (Bain et al., Langmuir, 5:723-727 (1989)) and silver (Xia et al., Langmuir, 22:269, (1998)). In general, the problem of attaching biologically active molecules to the surface of a substrate, whether it is a metal electrical conductor or an electrical insulator such as glass, is more difficult than the simple chemical reaction of a reactive group on the biological molecule with a complementary reactive group on the substrate. For example, a metal electrical conductor has no reactive sites, in principle, except those that may be adventitiously or deliberately positioned on the surface of the metal. Therefore, it would be desirable to have a way of controlling the attachment of different probes to different electrical conductors in order to provide an efficient means of detection of very small amounts of target nucleic acid molecules.

The present invention is directed to achieving these objectives.

### SUMMARY OF THE INVENTION

The present invention relates to a method of attaching nucleic acid molecules to electrically conductive surfaces. The method involves providing first and second electrical conductors, located near but not in contact with one another, where the first electrical conductor is made of a first type of conductive material and the second electrical conductor is made of a second type of conductive material which is different than the first type of conductive material. Next, a first set of oligonucleotide probes is attached to the first electrical conductor with an attachment chemistry which binds the first set of oligonucleotide probes to the first electrical conductor but not to the second electrical conductor. A second set of oligonucleotide probes is then attached to the second electrical conductor.

Another aspect of the present invention relates to a method of attaching nucleic acid molecules to electrically conductive surfaces. The method involves providing first and second electrical conductors located near, but not in contact with, one another, where the second electrical conductor is covered with a masking agent. Next, a first set of oligonucleotide probes is attached to the first electrical conductor with an attachment chemistry which binds the first set of oligonucleotide probes to the first electrical conductor. Then, the masking agent is removed from the second electrical conductor. Finally, a second set of oligonucleotide probes is attached to the second electrical conductor with an attachment chemistry which binds the second set of oligonucleotide probes to the second electrical conductor.

Yet another aspect of the present invention relates to a method of attaching multiple oligonucleotide probe molecules to electrically conductive surfaces. The method involves providing first and second electrical conductors, located near but not in contact with one another. Next, metal particles are attached to the first electrical conductor by silanizing a surface of the first electrical conductor and linking the silanized surface to the metal particles with a siloxane group. Multiple oligonucleotide probe molecules are then attached to the metal particles attached to the first electrical conductor.

The present invention also relates to a method of attaching nucleic acid molecules to electrically conductive surfaces. The method involves providing first and second electrical conductors located near, but not in contact with one another, where a voltage source is connected to the electrical conductors. A first set of oligonucleotide probes is then attracted toward the first electrical conductor by making the first electrical conductor more positively charged relative to the second electrical conductor, where the first set of oligonucleotide probes chemically binds to the first electrical conductor.

The present invention also relates to an apparatus for detecting a target nucleic acid molecule in a sample. The apparatus includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors.

Another aspect of the present invention relates to a method for detecting a target nucleic acid molecule in a sample. The method first involves providing an apparatus which includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors and spaced apart from the first set of oligonucleotide probes by a gap. Next, the probes are contacted with a sample potentially containing a target nucleic acid molecule under conditions effective to permit any of the target nucleic acid molecule in the sample to hybridize to both of the spaced apart oligonucleotide probes to bridge the gap and electrically couple the pair of oligonucleotide probes with the hybridized target nucleic acid molecule, if any. The electrically coupled pair of oligonucleotide probes and the hybridized target nucleic acid molecule are then filled with a filling nucleic acid sequence, where the filling nucleic acid sequence is complementary to the target nucleic acid molecule and extends between the pair of oligonucleotide probes. Finally, it is determined if an electrical current can be carried between the probes, where the electrical current between the probes indicates the presence of the target nucleic acid molecule in the sample which has sequences complementary to the probes.

Yet another aspect of the present invention relates to a method for detecting a target nucleic acid molecule in a sample. The method first involves providing an apparatus which includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors and spaced apart from the first set of oligonucleotide probes by a gap. Next, the probes are contacted with a sample potentially containing a target nucleic acid molecule under conditions effective to permit any of the target nucleic acid molecule in the sample to hybridize to both of the spaced apart oligonucleotide probes to bridge the gap and electrically couple the pair of oligonucleotide probes with the hybridized target nucleic acid molecule, if any. A conductive material is then applied over the electrically coupled pair of oligonucleotide probes and the hybridized target nucleic acid molecule. Finally, it is determined if an electrical current can be carried between the probes, where the electrical current between the probes indicates the presence of the target nucleic acid molecule in the sample which has sequences complementary to the probes.

The present invention not only provide a means of attaching two different nucleic acid molecules to two different electrical conductors in a DNA detection device, but allows sensitive DNA detection devices to be fabricated at a lower cost.

### BRIEF DESCRIPTION OF THE DRAWINGS

FIG. 1A depicts an apparatus of the present invention where oligonucleotide probes are attached to electrical conductors in the form of spaced part conductive fingers. FIG. 1B shows how a target nucleic acid molecule present in a sample is detected by the apparatus.

FIG. 2 depicts a side view of an apparatus of the present invention with two electrical conductors made of different types of material, each having different attachment chemistry.

FIGS. 3A-E depict the sequence of steps that are necessary for attaching one kind of oligonucleotide probe to one electrical conductor and another kind of oligonucleotide probe to the other electrical conductor of FIG. 1.

FIG. 4 depicts a top view of an electrical conductor arrangement which is advantageously used when different populations of oligonucleotide probes are presented on different electrical conductors.

FIGS. 5A-F depict the sequence of steps that are necessary for attaching two different oligonucleotide probes to two different electrical conductors made of the same metal.

FIGS. 6A-D show the sequence of steps that are necessary for attaching multiple oligonucleotide probe molecules to an electrical conductor.

FIGS. 7A-C depict the sequence of steps that are necessary for attaching oligonucleotide probes to electrical conductors by electrostatically attracting the probes toward the electrical conductors.

FIGS. 8A-H show the sequence of steps that are necessary for electrostatically attaching oligonucleotide probes to electrical conductors by sequentially electroplating the electrical conductors with a specific metal.

### DETAILED DESCRIPTION OF THE INVENTION

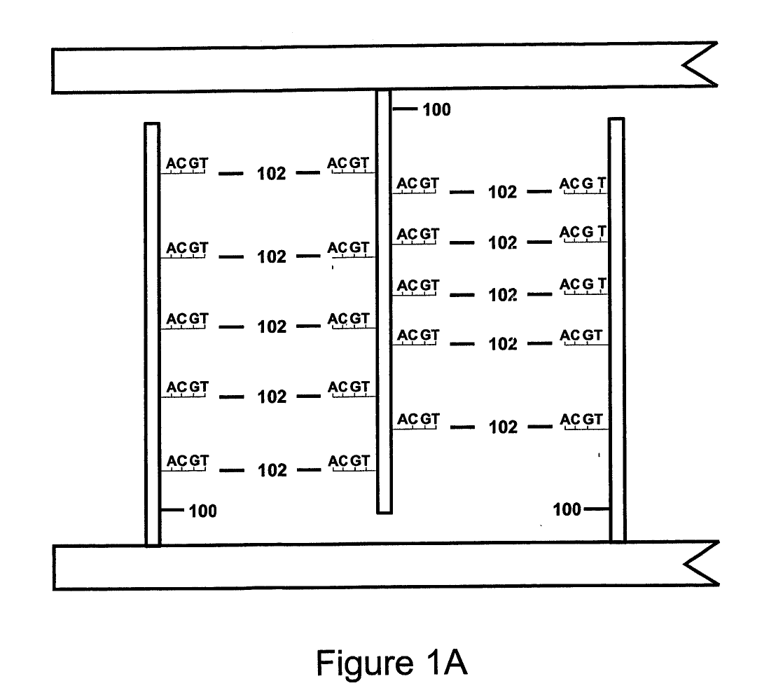

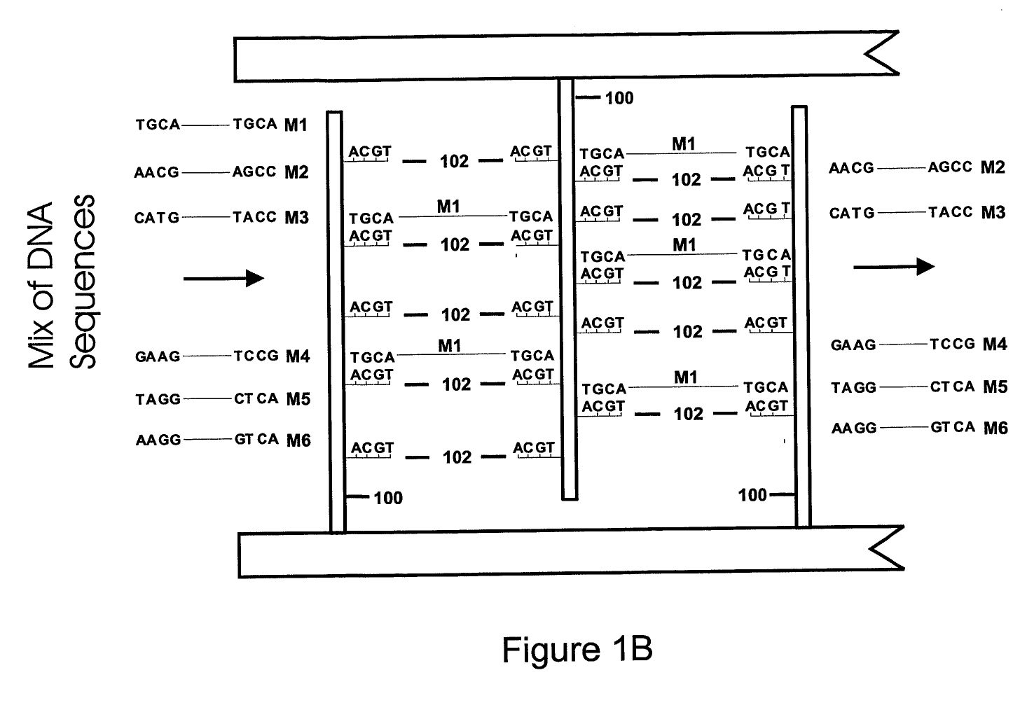

The present invention relates to the manufacture and use of a device which detects target nucleic acid molecules from samples. To put the present invention in perspective, this device and its use are shown in FIGS. 1A-B. According to FIG. 1A, oligonucleotide probes 102 attached to spaced apart conductive fingers 100 are physically located at a distance sufficient that they cannot come into contact with one another. A sample, containing a mixture of nucleic acid molecules (i.e. M1-M6), to be tested is contacted with the fabricated device on which conductive fingers 100 are fixed, as shown in FIG. 1B. If a target nucleic acid molecule (i.e. M1) that is capable of binding to the two oligonucleotide probes is present in the sample, the target nucleic acid molecule will bind to the two probe molecules. If bound, the nucleic acid molecule can bridge the gap between the two electrodes and provide an electrical connection. Any unhybridized nucleic acid molecules (i.e. M2-M6) not captured by the probes is washed away. Here, the electrical conductivity of nucleic acid molecules is relied upon to transmit the electrical signal. Hans-Werner Fink and Christian Schoenenberger reported in Nature, 398:407-410 (1999), which is hereby incorporated by reference in its entirety, that DNA conducts electricity like a semiconductor. This flow of current can be sufficient to construct a simple switch, which will indicate whether or not a target nucleic acid molecule is present within a sample. The presence of a target molecule can be detected as an “on” switch, while a set of probes not connected by a target molecule would be an “off” switch. The information can be processed by a digital computer which correlates the status of the switch with the presence of a particular target. The information can be quickly identified to the user as indicating the presence or absence of the biological material, organism, mutation, or other target of interest. Optionally, after hybridization of the target molecules to sets of biological probes, the target molecule can be coated with a conductor, such as a metal. The coated target molecule can then conduct electricity across the gap between the pair of probes, thus producing a detectable signal indicative of the presence of a target molecule.

One aspect of the present invention relates to a method of attaching nucleic acid molecules to electrically conductive surfaces. The method involves providing first and second electrical conductors, located near but not in contact with one another, wherein the first electrical conductor is made of a first type of conductive material and the second electrical conductor is made of a second type of conductive material which is different than the first type of conductive material. Next, a first set of oligonucleotide probes is attached to the first electrical conductor with an attachment chemistry which binds the first set of oligonucleotide probes to the first electrical conductor but not to the second electrical conductor. A second set of oligonucleotide probes is then attached to the second electrical conductor.

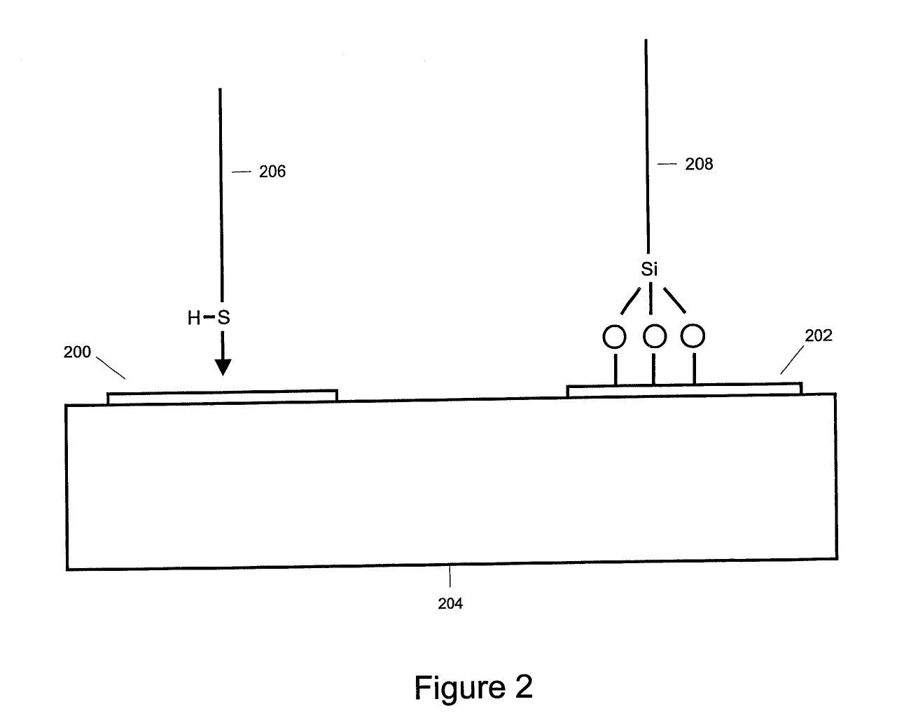

FIG. 2 depicts this aspect of the present invention, where first electrical conductor 200 and second electrical conductor 202 have different attachment chemistries for binding oligonucleotide probes to the electrical conductors. First oligonucleotide probe 206 is attached to first electrical conductor 200 by a dative bond, represented by an arrow, between the mercapto termination of first oligonucleotide probe 206 and the surface of first electrical conductor 200. Second oligonucleotide probe 208 is attached to second electrical conductor 202 by a siloxane bond to the surface of second electrical conductor 202. The first and second electrical conductors are fixed on substrate 204. Examples of useful substrate materials include glass, quartz and silicon as well as polymeric material such as plastics.

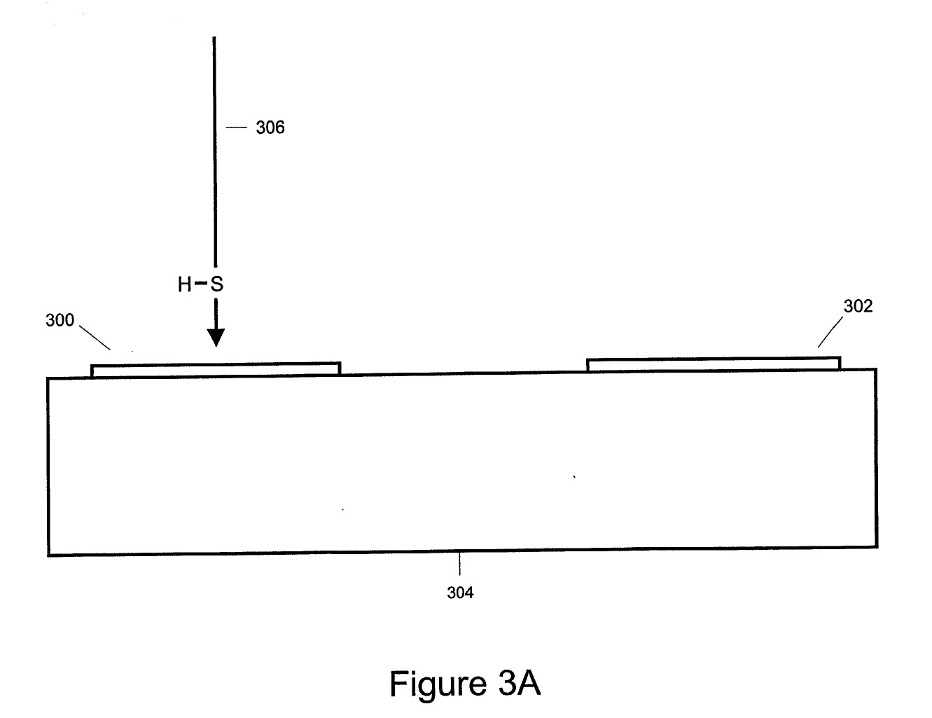

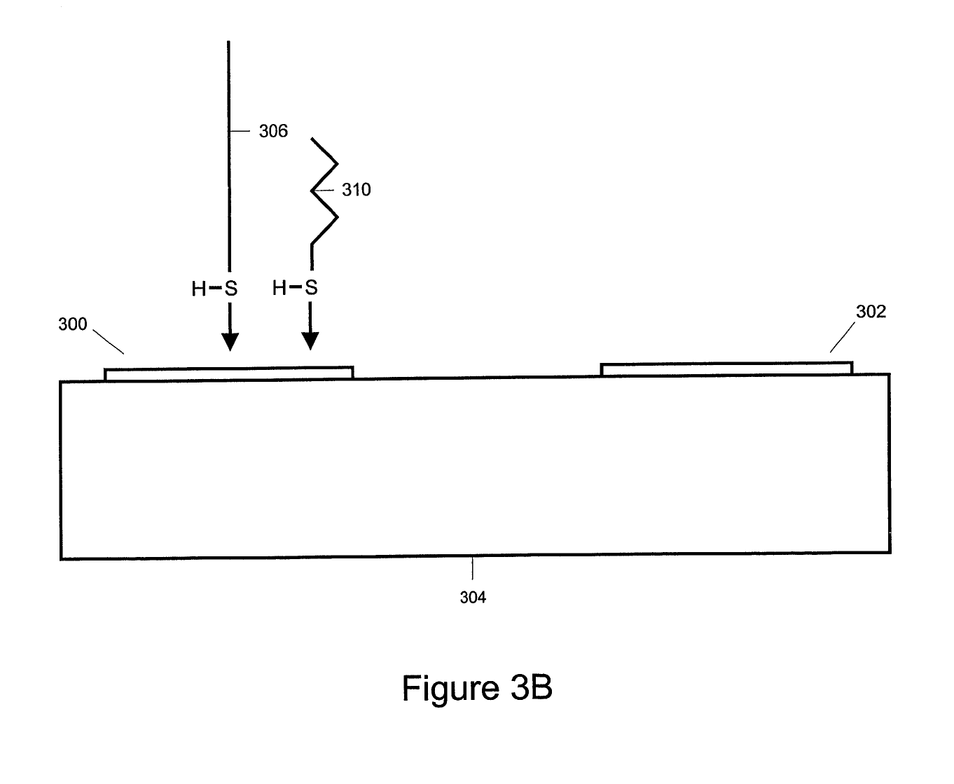

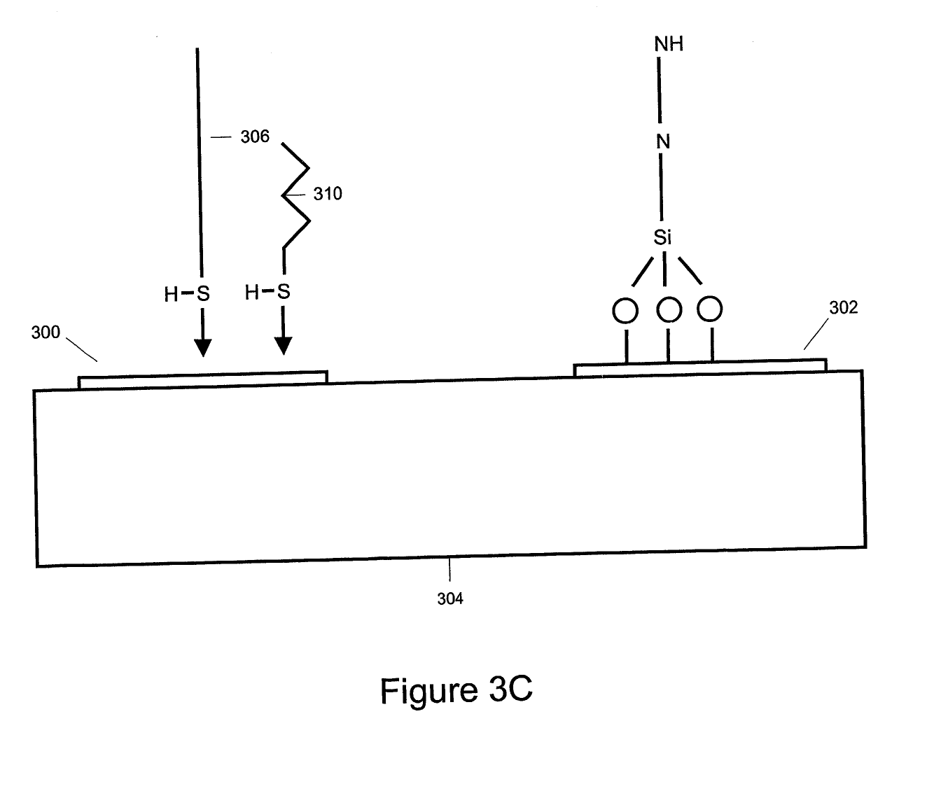

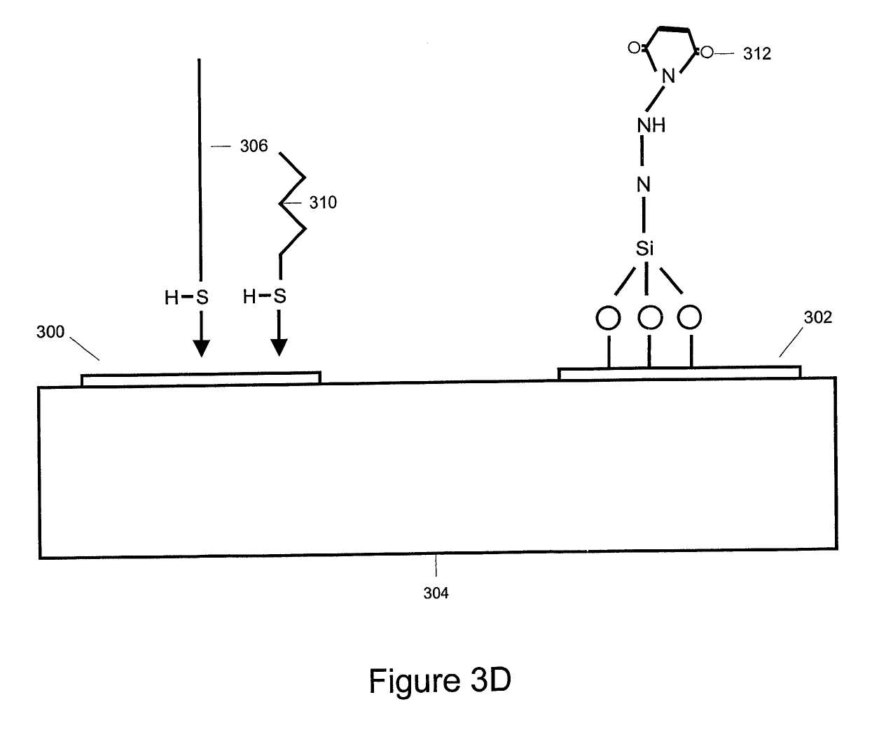

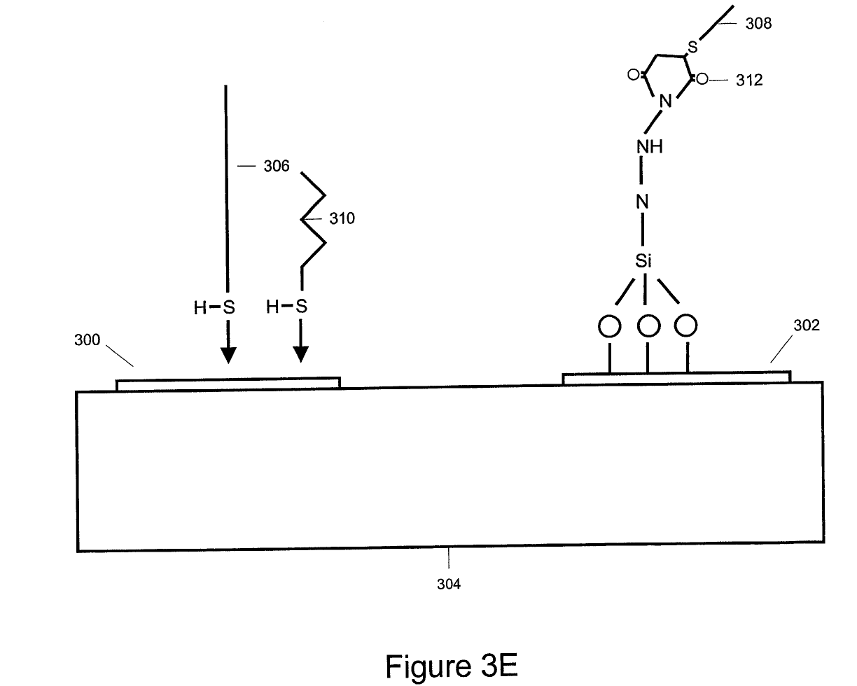

FIGS. 3A-F illustrate the sequence of steps necessary for attaching one kind of oligonucleotide probe to one electrical conductor and another kind of oligonucleotide probe to another electrical conductor where the two electrical conductors have different attachment chemistries. FIG. 3A shows the attachment of first oligonucleotide probe 306 to first electrical conductor 300. As described above, this attachment is accomplished by bathing the electrical conductor with a solution of the oligonucleotide probe in a suitable solvent. First oligonucleotide probe 306 does not attach to second electrical conductor 302, because the second electrical conductor does not have the suitable attachment chemistry. In FIG. 3B, all remaining sites on first electrical conductor 300 are blocked by bathing the electrical conductor in a solution of blocking molecules 310, represented by a zigzag line. FIG. 3C shows the surface of second electrical conductor 302, after silanization of the surface with N-[3-(trimethoxysilyl)propyl]ethylenediamine. FIG. 3D shows second electrical conductor 302 with linker molecule 312 attached to the siloxane. FIG. 3E shows the attachment of second oligonucleotide probe 308 to linker molecule 312 bound to second electrical conductor 302.

In one embodiment of this aspect of the present invention, after attaching a first set of oligonucleotide probes and before attaching a second set of oligonucleotide probes, blocking molecules are attached to the first electrical conductor at all sites not occupied by the first set of oligonucleotide probes. The blocking molecules will prevent nonspecific DNA binding as well as prevent any more oligonucleotide probes from binding. An example of a blocking molecule is dodecanethiol, a highly effective reagent for covering the surface of gold or silver with a self-assembled monolayer (SAM) of dodecanethiol. The effectiveness of this reagent derives from the extra bonding energy of VanderWaals interactions of the closely-packed hydrocarbon chains extending from the surface of the gold. Whatever the mechanism, treatment of the first electrical conductor surface with blocking molecules prevents further bonding of oligonucleotide probes.

In another embodiment, after attaching blocking molecules and before attaching a second set of oligonucleotide probes, the surface of the second electrical conductor is functionalized to permit the second set of oligonucleotide probes to be attached to the second electrical conductor. The surface of the second electrical conductor can be functionalized with hydroxyl groups. For example, a freshly sputtered aluminum surface does not wet well with water. That is, the contact angle formed by a drop of pure water is high and the water beads up and runs off the aluminum surface, rather than spreading and covering the surface of the aluminum. This is indicative of a surface with few hydroxyl groups. In order to increase the number of hydroxyl groups on the surface of the aluminum to provide reactive sites for the attachment chemistry, the aluminum electrical conductor can be cleaned by submersing the surface in a mixture of 10 parts of 30% hydrogen peroxide with about 1 part to 4 parts of concentrated ammonia. The aluminum is incubated in the mixture at room temperature for 15 to 30 minutes, then rinsed several times with pure water and dried. A check with a small drop of water shows that the water spreads and wets the surface, indicating that the number of hydroxyl groups has been increased. These hydroxyl groups provide reaction sites for attachment of oligonucleotide probes.

In another embodiment of the present invention, the first type of conductive material is gold, the second type of conductive material is aluminum, the attachment chemistry for the first electrical conductor is a mercapto group, and the blocking molecules have thiol groups which are attached to the first electrical conductor.

While electrical conductors made of gold or aluminum have been mentioned, it is possible to use other materials as well. For example, metals, such as titanium, tantalum, chromium, copper, and zinc, can be used as electrical conductors. Although most electrically conductive electrical conductors are composed of metallic elements, either singly or in combination, it is also possible to use other non-metallic electrically conductive materials. For example, indium tin oxide (ITO) is commonly used as a transparent conductor in such devices as portable computer monitors. Silicon in pure form is a semi-conductor, but can be doped with materials, such as boron, to provide sufficient conductivity for use as an electrical conductor.

There are few examples of effective molecular attachment to electrically conducting surfaces except for gold (Bain et al., Langmuir, 5:723-727 (1989), which is hereby incorporated by reference in its entirety) and silver (Xia et al., Langmuir, 22:269, (1998), which is hereby incorporated by reference in its entirety). Attachment of a mercapto-terminated oligonucleotide probe to a gold electrical conductor can be accomplished by merely bathing the gold electrical conductor in a solution of the oligonucleotide probe molecules in a suitable solvent, such as water or dimethylsulfoxide, for about 1 to 5 minutes, followed by a rinse with the same solvent. Bonding occurs through the formation of a dative bond between the sulfur and gold atoms.

In another embodiment, the second set of oligonucleotide probes is attached to the second electrical conductor by silanizing a surface of the second electrical conductor and linking the silanized surface of the second electrical conductor to the second set of oligonucleotide probes with a siloxane group. This can be accomplished, in the case of an aluminum electrical conductor, by cleaning the aluminum surface with a mixture of hydrogen peroxide and ammonium hydroxide. The cleaned, hydroxylated aluminum electrical conductor is then treated with a toluene solution of a trialkoxysilane. Preferably, N-[3-(trimethoxysilyl)propyl]ethylenediamine, sold as Z-6094 (Dow Corning Company, Midland, Mich.) is dissolved in toluene at a concentration from about 1 part per 10,000 to 1 part per 100 parts of toluene, and preferably at a concentration of about 1 part per 1000 parts of toluene. The toluene solution is used to soak the aluminum surface for 15 minutes at room temperature. The aluminum is then rinsed with toluene and dried in air.

The silanized aluminum surface can then be soaked in a solution of a linker molecule in a dipolar aprotic solvent such as methyl sulfoxide or dimethylformamide. The linker molecule terminates on one end with a group reactive toward primary amines, and at the other end with a group reactive toward thiols. Examples of such linker molecules are N-(α-maleimidoacetoxyl)succinimide ester, N-(β-maleimidopropyloxy)succinimide ester, N-(γ-maleimidobutyryloxy)succinimide ester, succinimidyl-4-(N-maleimidomethyl)-cyclohexane-1-carboxy-(6-amiidocaproate), m-maleimidobenzoyl-N-hydroxysuccinimide ester, N-succinimidyl iodoacetate, and N-succinimidyl-(4-vinylsulfonyl)benzoate, all sold by Pierce Company (Rockford, Ill.). The linker molecule can be used at a concentration of from about 0.1% (by weight) to about 10% in dimethylsulfoxide or dimethylformamide, and more preferably, at a concentration of about 1%. The surface of the silanized aluminum electrical conductor is bathed in the linker solution for from about 1 minute to 60 minutes, and more preferably from about 10 to 20 minutes. The electrical conductor is then rinsed with the same solvent followed by a water rinse and allowed to air dry.

Finally, an oligonucleotide probe terminated at either the 3′ or 5′ end with a mercapto group in water or an aqueous buffer, such as a 0.1 M solution of sodium phosphate in water, can be used to coat or submerge the electrical conductor to cause the reaction of the maleimide end of the linker with the mercapto group to form a thiol ether covalent bond between the oligonucleotide probe and the linker. The oligonucleotide probe is used at a concentration of from about one picogram per microliter to about one microgram per microliter. A dipolar aprotic solvent such as dimethylsulfoxide or dimethylformamide may also be used instead of water to dissolve the oligonucleotide probe. The probe solution is used to bathe the electrical conductor for from about 1 minute to 60 minutes, and more preferably from about 10 to 20 minutes. The electrical conductor is then rinsed with the same solvent followed by a water rinse and allowed to air dry. The electrical conductor is then ready for hybridization with a target nucleic acid molecule.

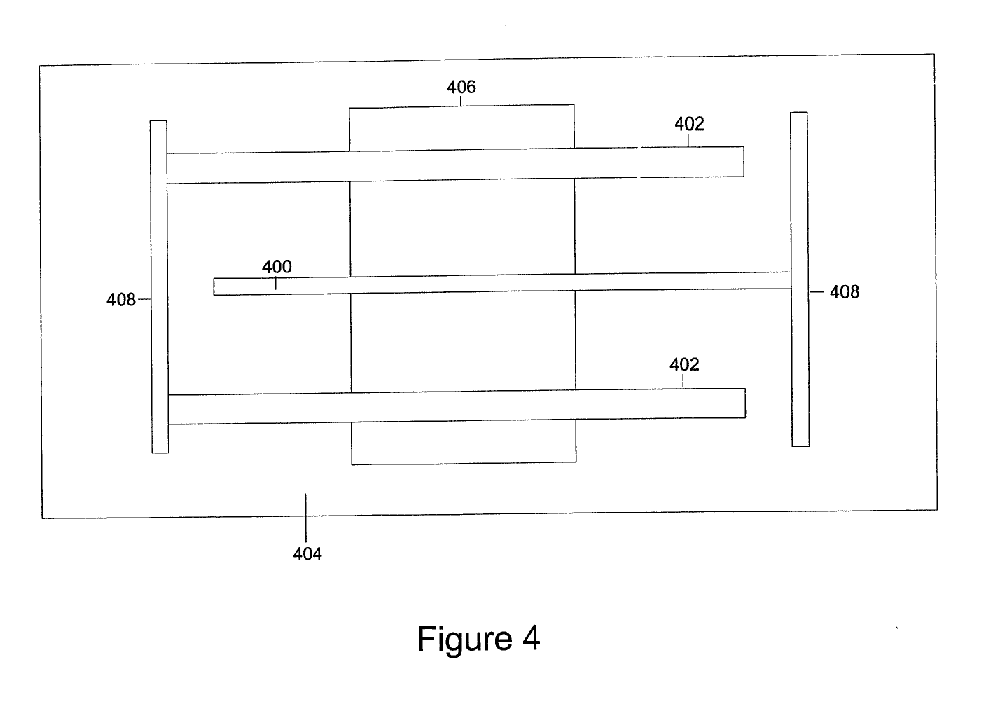

FIG. 4 shows the top view of an electrical conductor arrangement that can be advantageously used when one probe has a higher population than the other probe. Thus, thinner, central first electrical conductor 400 is flanked on both sides by wider second electrical conductors 402. The electrical conductors are deposited on insulating substrate 404. A heavy black line outlines active area boundary 406 of the device. Electrical contact pads 408 for electrical contact are shown as vertical rectangles. Since there are more probe molecules on first electrical conductor 400, hybridization of the target nucleic acid molecule has a high probability of occurring first on first electrical conductor 400. Thus, one end of the target nucleic acid molecule is tethered to first electrical conductor 400, and the other free end of the target nucleic acid molecule can explore the larger area of second electrical conductor 402, where the second probes are attached, over a relatively long length of time without escaping, thereby increasing the probability of hybridizing with the second probe.

Alternatively, it may be preferable to construct both electrical conductors from the same type of material. This can be achieved by using a masking agent. Thus, another aspect of the present invention relates to a method of attaching nucleic acid molecules to electrically conductive surfaces. The method involves providing first and second electrical conductors located near, but not in contact with, one another, where the second electrical conductor is covered with a masking agent. Next, a first set of oligonucleotide probes is attached to the first electrical conductor with an attachment chemistry which binds the first set of oligonucleotide probes to the first electrical conductor. Then, the masking agent is removed from the second electrical conductor. Finally, a second set of oligonucleotide probes is attached to the second electrical conductor with an attachment chemistry which binds the second set of oligonucleotide probes to the second electrical conductor.

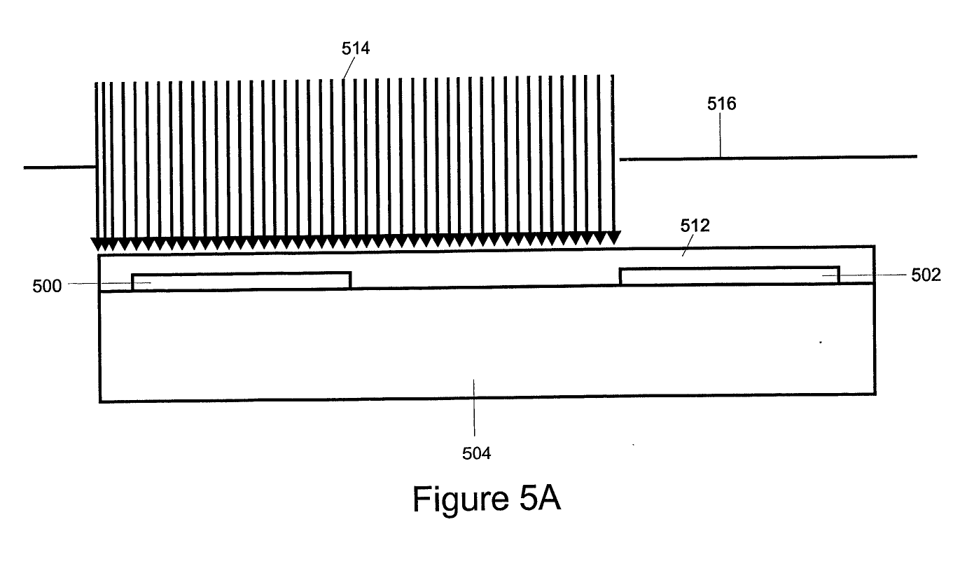

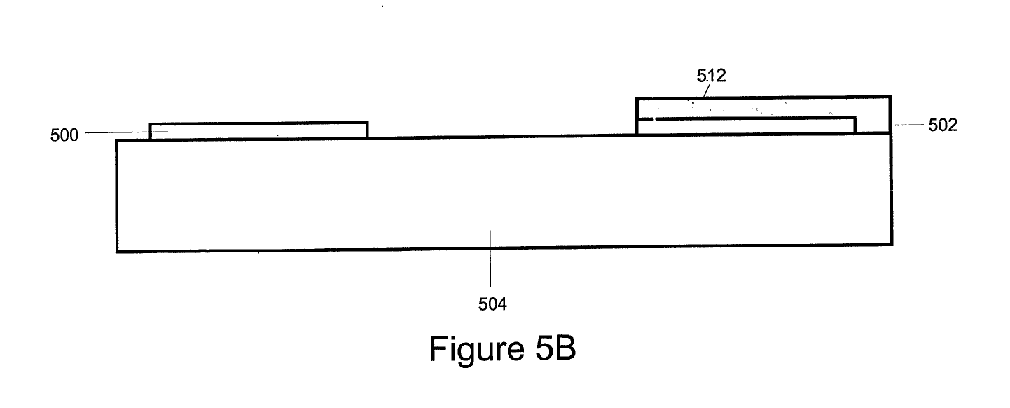

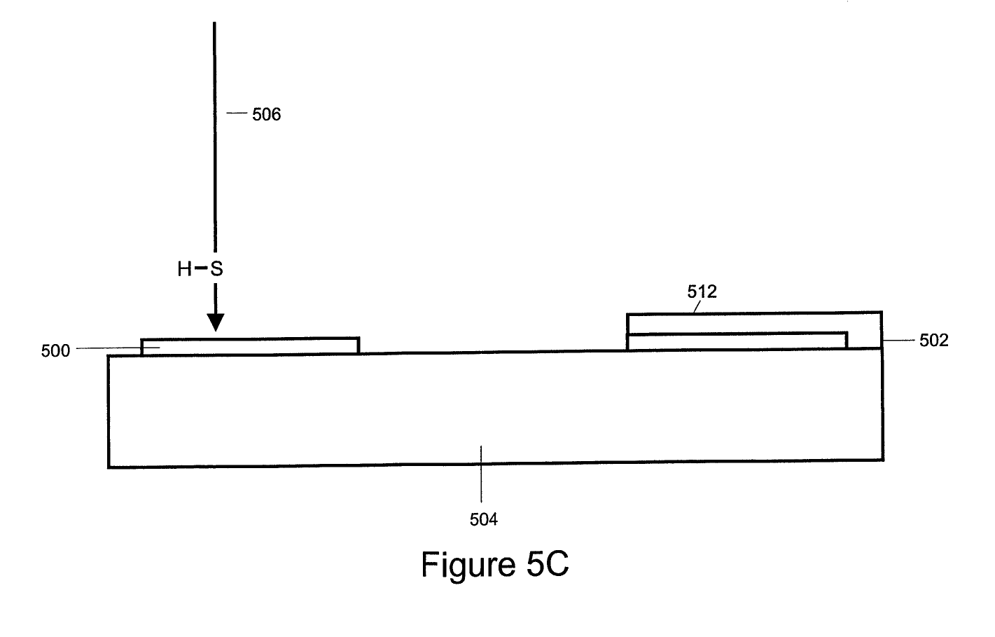

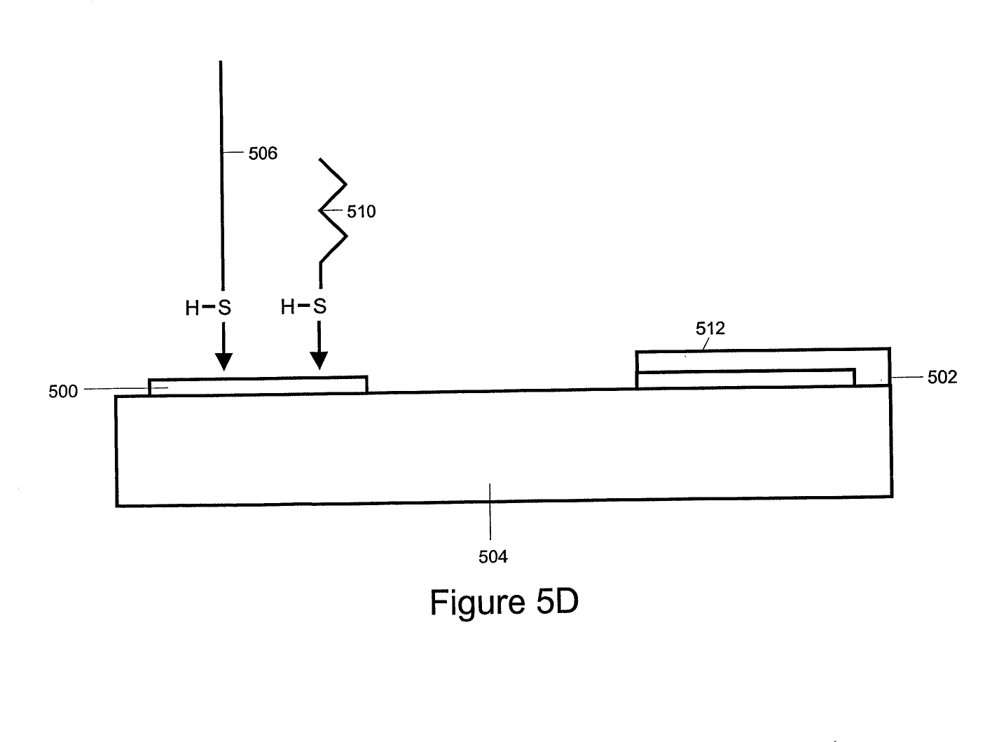

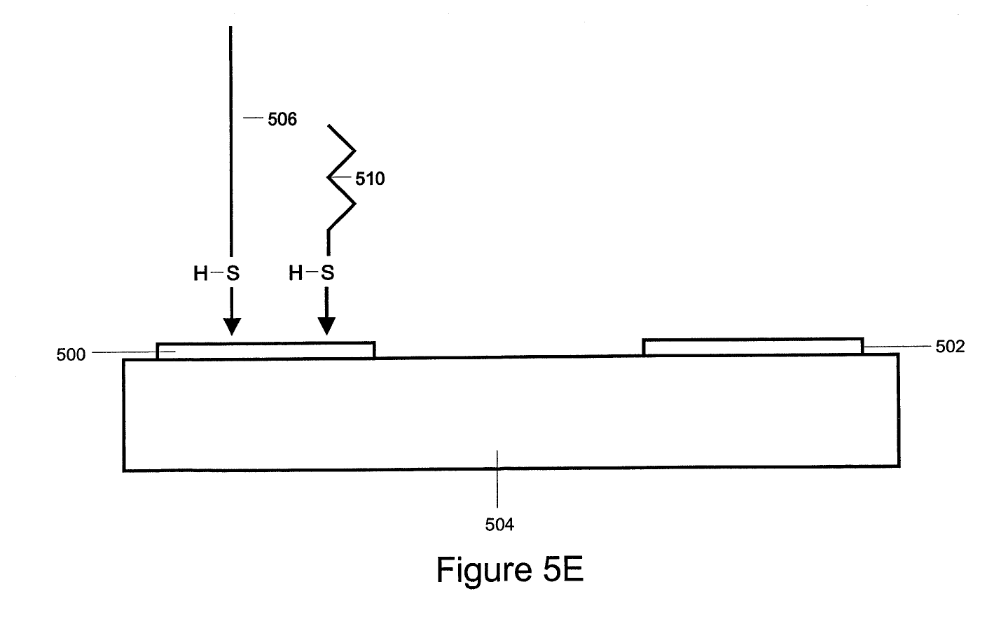

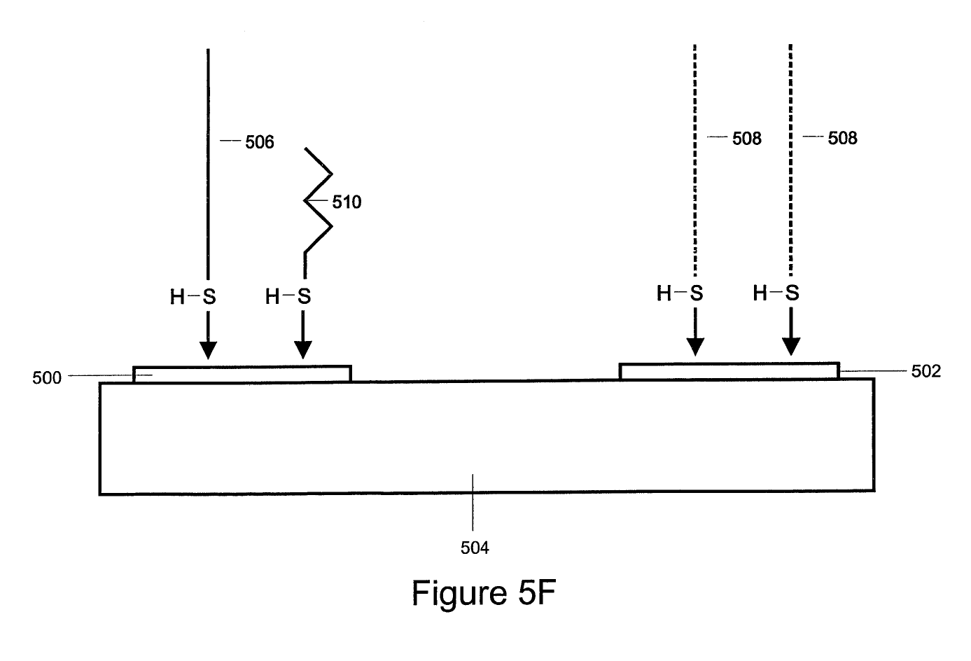

FIGS. 5A-F illustrate the sequence of steps for attaching two different oligonucleotide probe molecules to two electrical conductors made of the same material, where a masking agent is used. FIG. 5A shows first and second electrical conductors 500 and 502 made from the same metal covered with a layer of masking agent 512. First electrical conductor 500 is exposed to ultraviolet light 514, represented by arrows, through photolithographic mask 516. After exposure, a developer removes the exposed area of masking agent 512, the result of which is shown in FIG. 5B. Exposed first electrical conductor 500 is then bathed in a solution of a first oligonucleotide probe 506, shown as a thick line terminated with a mercapto group, resulting in the attachment of the probe as shown in FIG. 5C. First electrical conductor 500 is then bathed in a blocking solution of blocking molecules 510, represented by a zigzag line, to cover any remaining sites on exposed first electrical conductor 500, as shown in FIG. 5D. The remaining masking agent 512 is then removed with acetone, as shown in FIG. 5E. Second oligonucleotide probe 508 is then attached by bathing second electrical conductor 502 in a solution of second oligonucleotide probe 508, represented by a dotted line, as shown in FIG. 5F. First and second electrical conductors are fixed on substrate 504.

In one embodiment of this aspect of the present invention, after attaching a first set of oligonucleotide probes or attaching a second set of oligonucleotide probes, blocking molecules such as dodecanethiol can be attached to the first or second electrical conductors at all sites not occupied by the first or second set of oligonucleotide probes.

In another embodiment, the first and second electrical conductors are covered with a masking agent, and the masking agent is removed from the first electrical conductor but not from the second electrical conductor, prior to attaching a first set of oligonucleotide probes to the first electrical conductors. The masking agent may be a layer of polymer such as photoresist, or another metal or any other material as long as it could cover an electrical conductor and be selectively removable without disrupting the nucleic acid on the other electrical conductor. If the masking agent is photoresist, the photoresist can be removed from the first or second electrical conductor by exposing the photoresist at a location corresponding to the first or second electrical conductor with radiation and removing the exposed photoresist.

In another embodiment, the first and second electrical conductors are made of gold, the attachment chemistry for the first and second electrical conductors is a mercapto group, and the blocking molecules have thiol groups attached to the first and second electrical conductors.

Yet another aspect of the present invention relates to a method of attaching multiple oligonucleotide probe molecules to electrically conductive surfaces. The method involves providing first and second electrical conductors, located near but not in contact with one another. Next, metal particles are attached to the first electrical conductor by silanizing a surface of the first electrical conductor and linking the silanized surface to the metal particles with a siloxane group. Multiple oligonucleotide probe molecules are then attached to the metal particles attached to the first electrical conductor. Previous methods of attaching oligonucleotide probes to silanized surfaces utilized bi-functional linkers that couple a single probe molecule to the siloxane. In contrast, the present invention uses a metal particle as the linker, where multiple probe molecules can be attached per siloxane molecule, thereby increasing the density and number of probe molecules attached to the electrical conductor. A detection device with a higher density of probe molecules will have a greater probability and rate of capture of a target molecule.

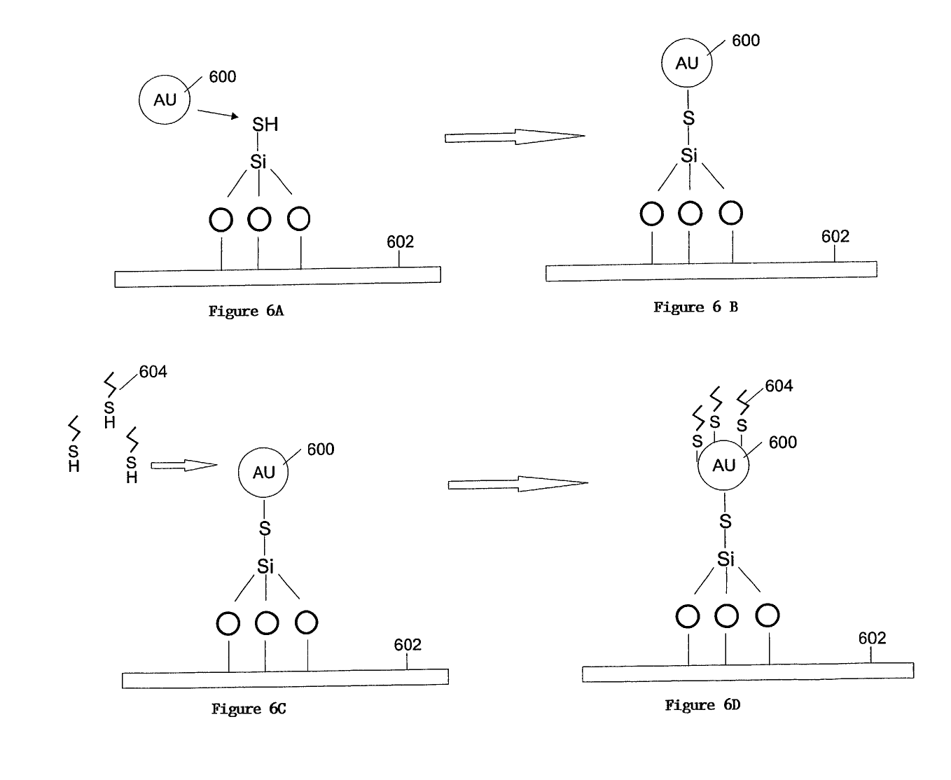

FIGS. 6A-D show one embodiment of this aspect of the present invention where the first electrical conductor is made of aluminum and the metal particles are made of gold. Gold particles of various desired sizes can be made as described in previously published methods. For example, reduction with a 1% gold chloride solution containing sodium citrate will generate 20 nm spherical gold particles. Gold particles 600 can bind to the thiol groups presented by aluminum electrical conductor 602 that has been coated with mercapto-siloxane, as illustrated in FIGS. 6A-B. Subsequently, oligonucleotide probe molecules 604 bind to the gold particles 600 through their own thiol moieties, as illustrated in FIGS. 6C-D.

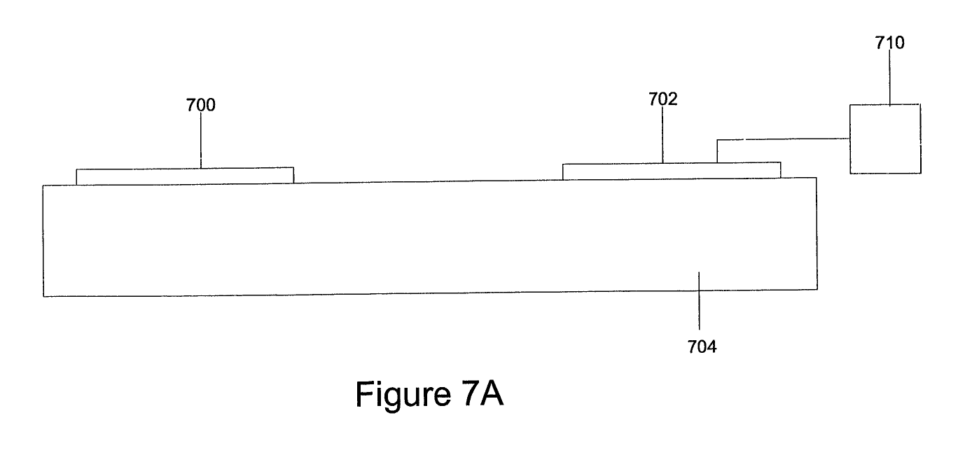

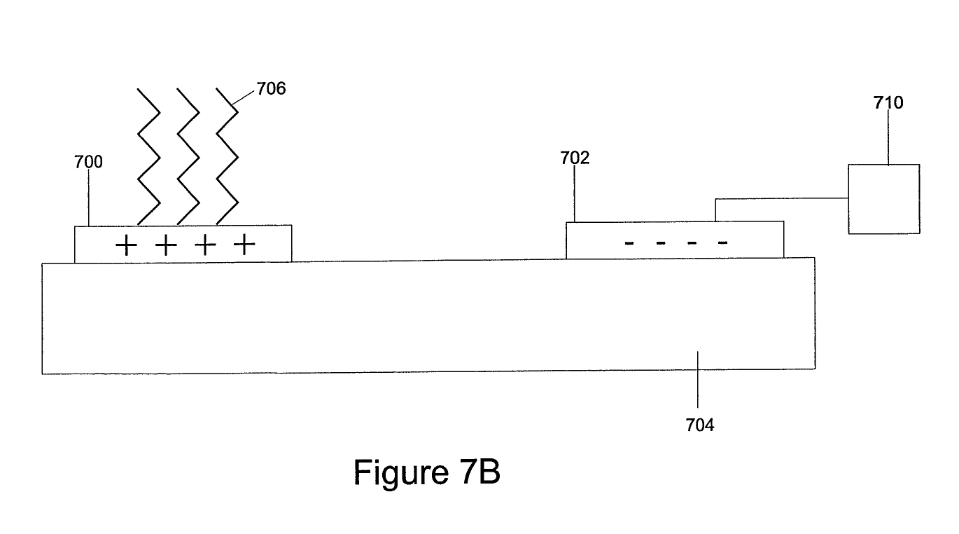

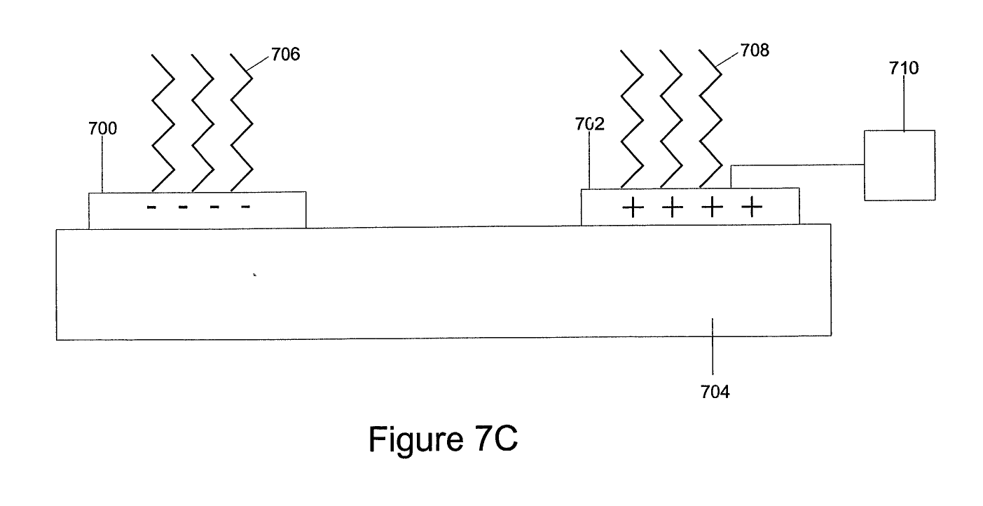

The present invention also relates to a method of attaching nucleic acid molecules to electrically conductive surfaces, where a charge is built up on the electrical conductor so that the electrical conductor electrostatically attracts oligonucleotide probes. The method involves providing first and second electrical conductors 700, 702 located near, but not in contact with one another, where voltage source 710 is connected to the electrical conductors, as shown in FIG. 7A. A first set of oligonucleotide probes 706 is then attracted toward first electrical conductor 700 by making the first electrical conductor 700 more positively charged relative to second electrical conductor 702, where the first set of oligonucleotide probes 706 chemically binds to first electrical conductor 700, as illustrated in FIG. 7B.

A second set of oligonucleotide probes 708 can be attracted toward second electrical conductor 702 by making second electrical conductor 702 more positively charged relative to first electrical conductor 700, where the second set of oligonucleotide probes 708 chemically binds to second electrical conductor 702, as shown in FIG. 7C.

In another embodiment, the first electrical conductor is positively charged and the second electrical conductor is negatively charged when attracting the first set of oligonucleotide probes, while the second electrical conductor is positively charged and the first electrical conductor is negatively charged when attracting the second set of oligonucleotide probes.

The first and second electrical conductors can be made of the same type of material.

In yet another embodiment, blocking molecules can be attached to the first electrical conductors at all sites not occupied by the first set of oligonucleotide probes after the first set of oligonucleotide probes binds to the first electrical conductor.

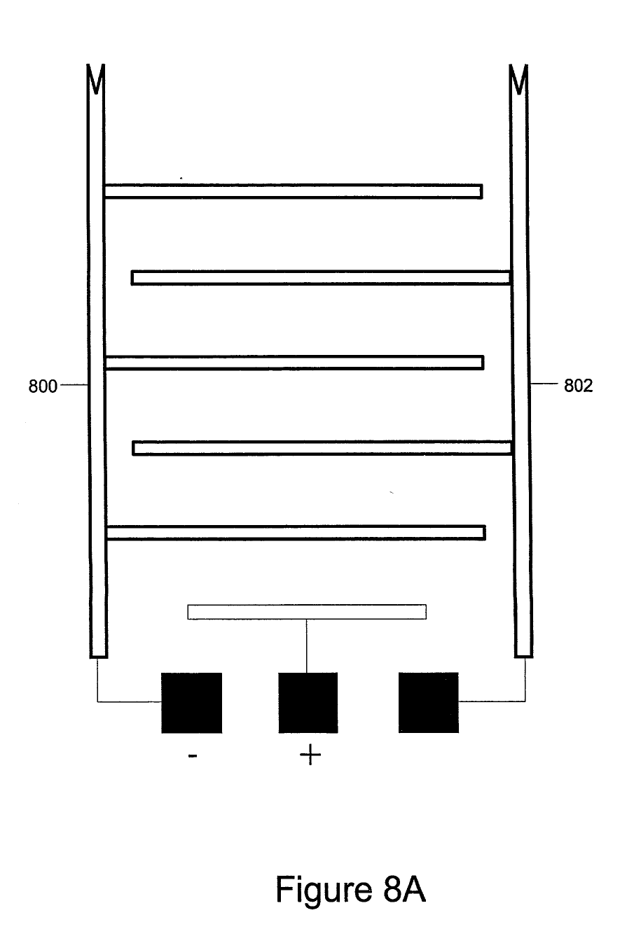

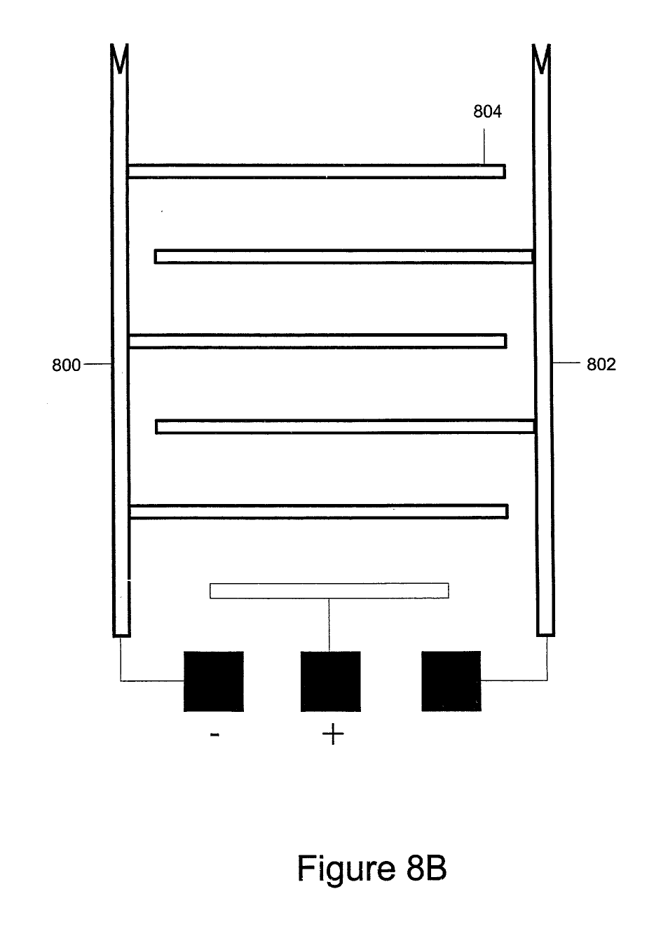

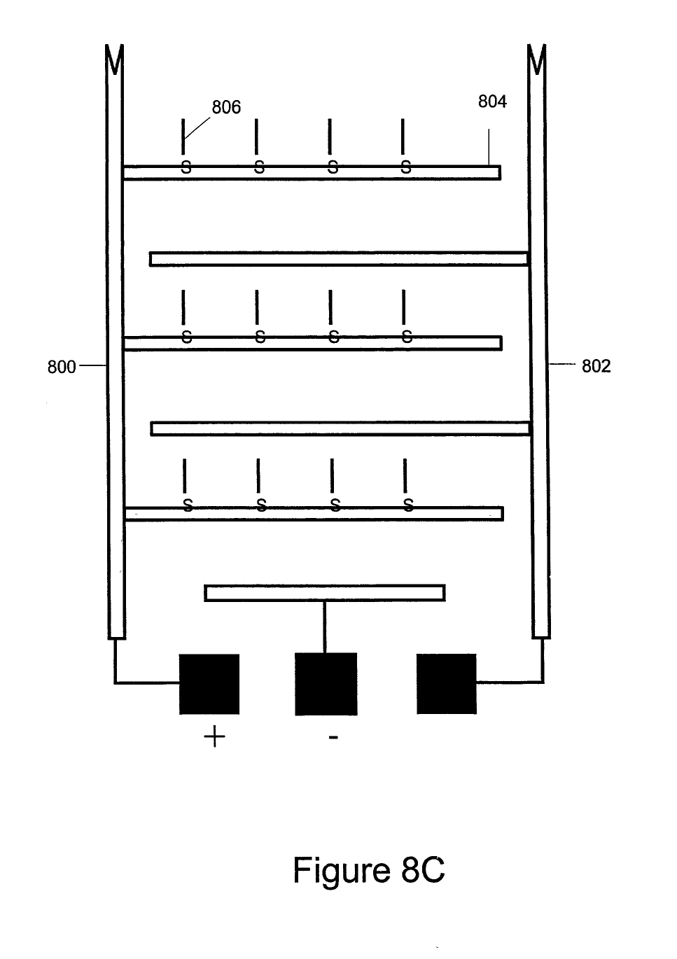

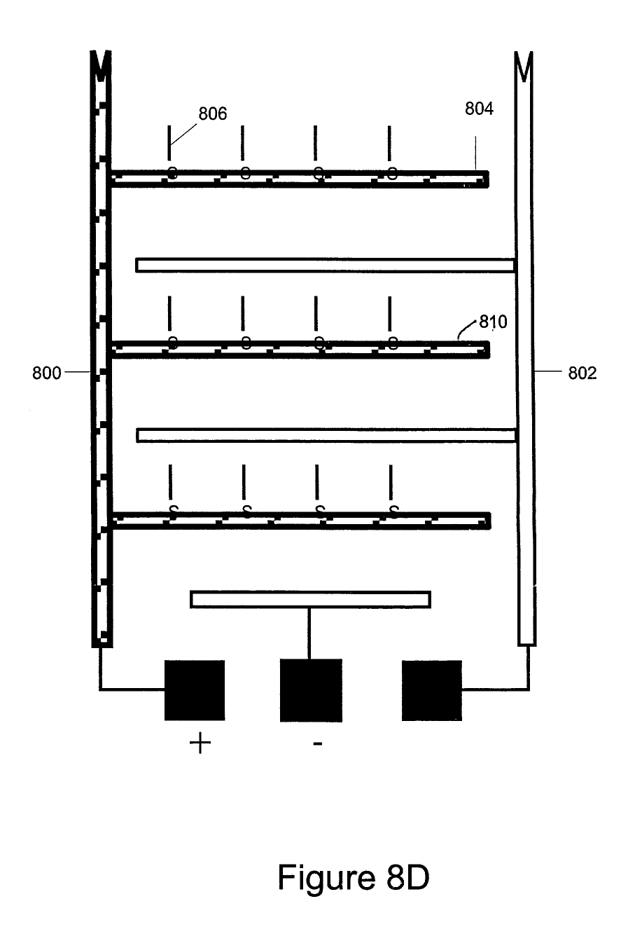

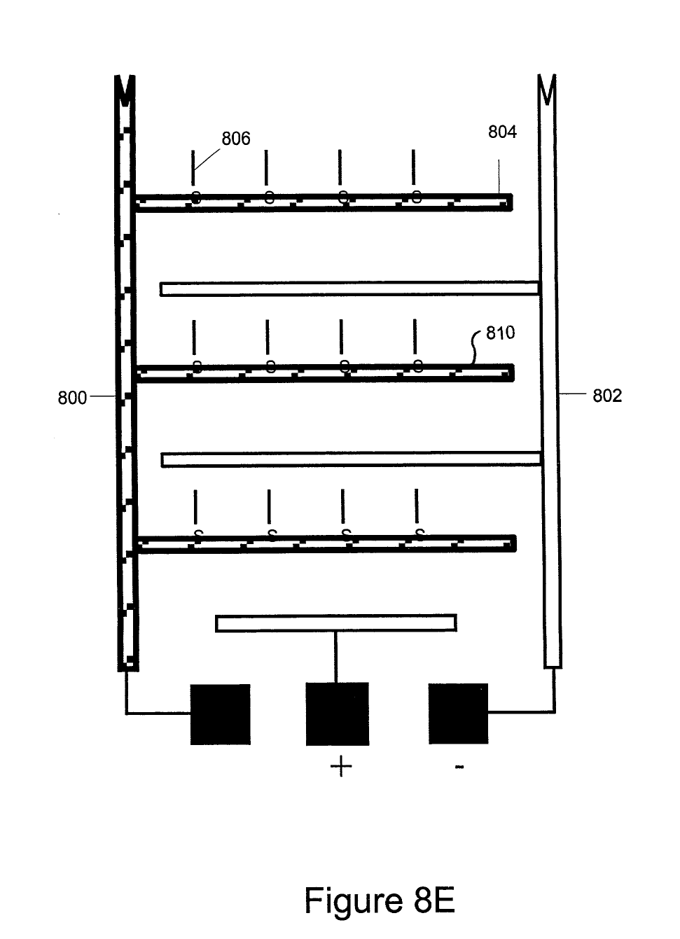

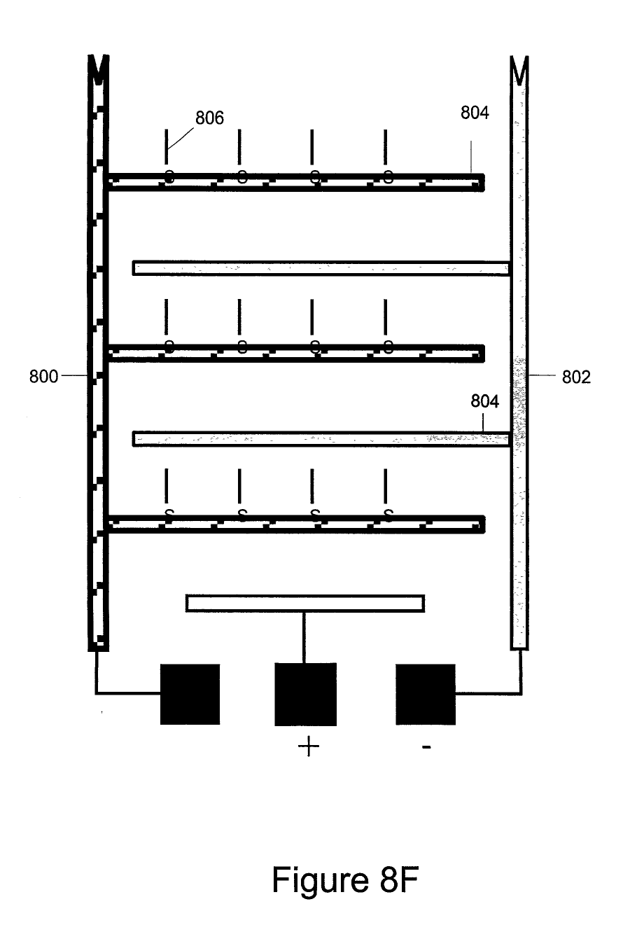

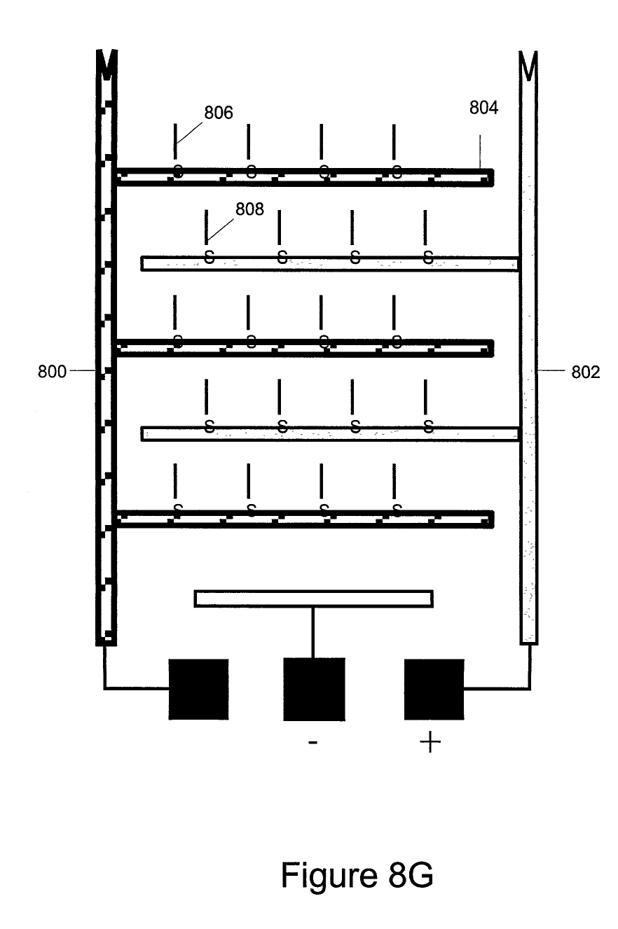

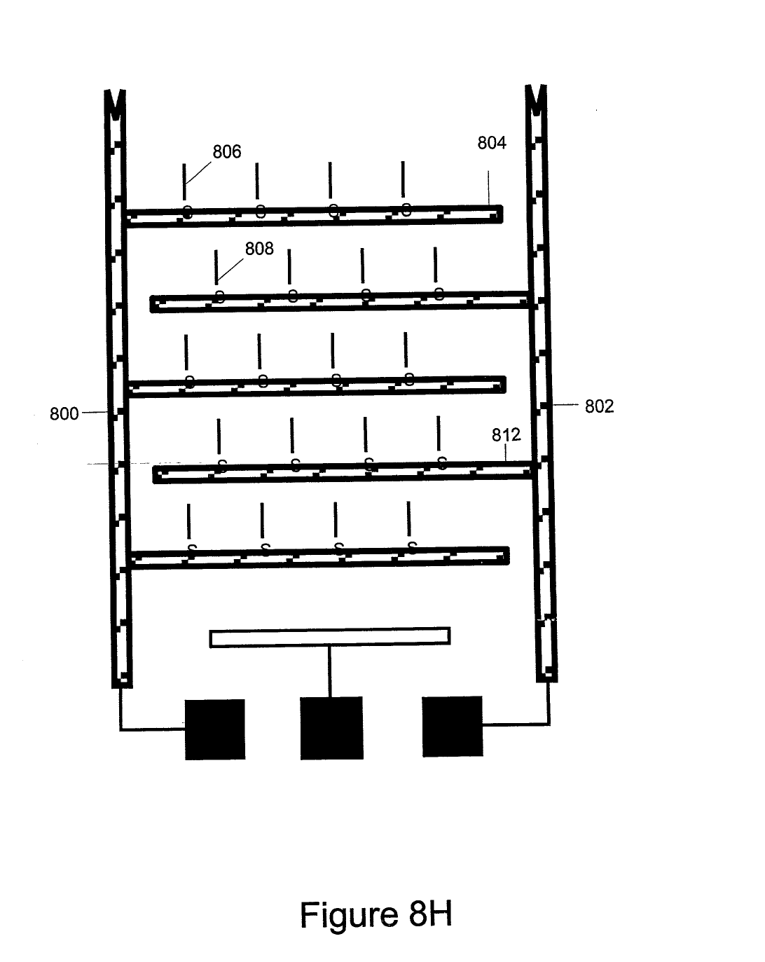

FIGS. 8A-F illustrate another embodiment of the present invention, which is an efficient method of directing different probe molecules to different electrical conductors by sequentially electroplating the electrical conductors with a specific metal and targeting thiol probe molecules to specific electrical conductors. First, prior to the step of attracting the first set of oligonucleotide probes, first electrical conductor 800 is electroplated with a specific metal 804 by placing the device in a electroplating solution and applying an electrical potential across the electrical conductors to electroplate a specific metal 804 onto first electrical conductor 800, as shown in FIGS. 8A-B. Next, the first set of oligonucleotide probes 806 which is negatively charged is attracted toward electroplated first electrical conductor 800 which is positively charged, as shown in FIG. 8C. Then, blocking molecules 810 are attached to first electrical conductor 800 at all sites not occupied by the first set of oligonucleotide probes 806, as shown in FIG. 8D. No electrical potential is needed for this step. Next, the device is placed back into the electroplating solution and an electrical potential opposite to the one applied earlier is applied to electroplate second electrical conductor 802 with a specific metal 804, as shown in FIGS. 8E-F. Then, the second set of oligonucleotide probes 808 which is negatively charged is attracted toward electroplated second electrical conductor 802 which is positively charged, as shown in FIG. 8G. The binding of the second set of oligonucleotide probes 808 is specific to second electrical conductor 802, because the electroplating on first electrical conductor 800 is occluded by blocking agent 812 and because the charge bias will concentrate the second set of oligonucleotide probes 808 around second electrical conductor 802. Then, blocking molecules 812 are attached to second electrical conductor 802 to prevent nonspecific binding of DNAs or RNAs, as shown in FIG. 8H. By having different oligonucleotide probe molecules specifically bound to opposite electrical conductors in the detection device, the unproductive binding of a target nucleic acid molecule's two complementary regions to oligonucleotide probes on the same electrical conductor will be reduced or eliminated, thereby increasing the sensitivity of the detection device.

The present invention also relates to an apparatus for detecting a target nucleic acid molecule in a sample. The apparatus includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors.

The first and second electrical conductors are fixed on a substrate. Examples of useful substrate materials include glass, quartz and silicon as well as polymeric substrates, e.g. plastics. In the case of conductive or semi-conductive substrates, it will generally be desirable to include an insulating layer on the substrate. However, any solid support which has a non-conductive surface may be used to construct the apparatus. The support surface need not be flat. In fact, the support may be on the walls of a chamber in a chip.

As chip manufacturing has improved, it has become possible to shrink the distance between the detection sites of the two electrical conductors on a chip. Thus, in one embodiment of this invention, the detection sites are located less than 100 microns apart. In another embodiment, the detection sites are located less than 10 microns apart.

Improved methods of forming large arrays of oligonucleotides, peptides and other polymer sequences with a minimal number of synthetic steps are known. See, U.S. Pat. No. 5,143,854 to Pirrung et al. (see also, PCT Application No. WO 90/15070) and Fodor et al., PCT Publication No. WO 92/10092, which are hereby incorporated by reference in their entirety, which disclose methods of forming vast arrays of peptides, oligonucleotides and other molecules using, for example, light-directed synthesis techniques. See also, Fodor et al., Science, 251:767-77 (1991), which is hereby incorporated by reference in its entirety. These procedures for synthesis of polymer arrays are now referred to as VLSIPS™ procedures.

Methods of synthesizing desired oligonucleotide probes are known to those of skill in the art. In particular, methods of synthesizing oligonucleotides and oligonucleotide analogues can be found in, for example, Oligonucleotide Synthesis: A Practical Approach, Gait, ed., IRI Press, Oxford (1984); Kuijpers, Nucleic Acids Research, 18(17):5197 (1994); Dueholm, J. Org. Chem., 59:5767-5773 (1994); and Agrawal (ed.), Methods in Molecular Biology, 20, which are hereby incorporated by reference in their entirety. Shorter oligonucleotide probes have lower specificity for a target nucleic acid molecule, that is, there may exist in nature more than one target nucleic acid molecule with a sequence of nucleotides complementary to the oligonucleotide probe. On the other hand, longer oligonucleotide probes have decreasingly smaller probabilities of containing complementary sequences to more than one natural target nucleic acid molecule. In addition, longer oligonucleotide probes exhibit longer hybridization times than shorter oligonucleotide probes. Since analysis time is a factor in a commercial device, the shortest possible probe that is sufficiently specific to the target nucleic acid molecule is desirable. Both the speed and specificity of binding target nucleic acid molecules to oligonucleotide probes can be increased if one electrical conductor has attached a probe that is complementary to one end of the target nucleic acid molecule and the other electrical conductor has attached a probe that is complementary to the other end of the target nucleic acid. In this case, even if short oligonucleotide probes that exhibit rapid hybridization rates are used, the specificity of the target nucleic acid molecules to the two probes is high. If two different probe molecules are used, it is important that both probes are not located on the same electrical conductor, to prevent hybridization of a target nucleic acid molecule from one part of an electrical conductor to another part of the same electrical conductor. If this happens, no signal can be generated from such an attachment, and the sensitivity of the analysis is lowered.

The present invention includes chemically modified nucleic acid molecules or oligonucleotide analogues as oligonucleotide probes. An “oligonucleotide analogue” refers to a polymer with two or more monomeric subunits, wherein the subunits have some structural features in common with a naturally occurring oligonucleotide which allow it to hybridize with a naturally occurring nucleic acid in solution. For instance, structural groups are optionally added to the ribose or base of a nucleoside for incorporation into an oligonucleotide, such as a methyl or allyl group at the 2′-O position on the ribose, or a fluoro group which substitutes for the 2′-O group, or a bromo group on the ribonucleoside base. The phosphodiester linkage, or “sugar-phosphate backbone” of the oligonucleotide analogue is substituted or modified, for instance with methyl phosphonates or O-methyl phosphates. Another example of an oligonucleotide analogue includes “peptide nucleic acids” in which native or modified nucleic acid bases are attached to a polyamide backbone. Oligonucleotide analogues optionally comprise a mixture of naturally occurring nucleotides and nucleotide analogues. Oligonucleotide analogue arrays composed of oligonucleotide analogues are resistant to hydrolysis or degradation by nuclease enzymes such as RNAase A. This has the advantage of providing the array with greater longevity by rendering it resistant to enzymatic degradation. For example, analogues comprising 2′-O-methyloligoribonucleotides are resistant to RNAase A.

Many modified nucleosides, nucleotides, and various bases suitable for incorporation into nucleosides are commercially available from a variety of manufacturers, including the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen, San Diego, Calif., and Applied Biosystems (Foster City, Calif.), as well as many other commercial sources known to one of skill. Methods of attaching bases to sugar moieties to form nucleosides are known. See, e.g., Lukevics and Zablocka, “Nucleoside Synthesis: Organosilicon Methods,” Ellis Horwood Limited Chichester, West Sussex, England (1991), which is hereby incorporated by reference in its entirety. Methods of phosphorylating nucleosides to form nucleotides, and of incorporating nucleotides into oligonucleotides are also known. See, e.g., Agrawal (ed), “Protocols for Oligonucleotides and Analogues, Synthesis and Properties,” Methods in Molecular Biology, volume 20, Humana Press, Towota, N.J. (1993), which is hereby incorporated by reference in its entirety.

The apparatus of the present invention can be used to detect target nucleic acid molecules in a sample. If a target nucleic acid molecule which contains sequences complementary to the first and second oligonucleotide probes is present in the sample, the target nucleic acid molecule makes a polymeric nucleotide connection between the two electrical conductors to complete an electrical circuit. Thus, the presence of a target nucleic acid molecule is indicated by the ability to conduct an electrical signal through the circuit. In the case where a target nucleic acid molecule is not present, the circuit will not be completed. Therefore, the target nucleic acid molecule acts as a switch. The presence of the nucleic acid molecule provides an “on” signal for an electrical circuit, whereas the lack of the target nucleic acid molecule is interpreted as an “off” signal. The information can be processed by a digital computer which correlates the status of the switch with the presence of a particular target. The computer can also analyze the results from several switches specific for the same target, to determine specificity of binding and target concentration.

In one embodiment, the native electrical conductivity of nucleic acid molecules can be relied upon to transmit the electrical signal. Fink et al. “Electrical Conduction through DNA Molecules,” Nature, 398:407-410 (1999), which is hereby incorporated by reference in its entirety, reported that DNA conducts electricity like a semiconductor. This flow of current can be sufficient to construct a simple switch. Thus, another aspect of the present invention relates to a method for detecting a target nucleic acid molecule in a sample. The method first involves providing an apparatus which includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors and spaced apart from the first set of oligonucleotide probes by a gap. Next, the probes are contacted with a sample potentially containing a target nucleic acid molecule under conditions effective to permit any of the target nucleic acid molecule in the sample to hybridize to both of the spaced apart oligonucleotide probes to bridge the gap and electrically couple the pair of oligonucleotide probes with the hybridized target nucleic acid molecule, if any. The electrically coupled pair of oligonucleotide probes and the hybridized target nucleic acid molecule are then filled with a filling nucleic acid sequence, where the filling nucleic acid sequence is complementary to the target nucleic acid molecule and extends between the pair of oligonucleotide probes. Finally, it is determined if an electrical current can be carried between the probes, where the electrical current between the probes indicates the presence of the target nucleic acid molecule in the sample which has sequences complementary to the probes.

Alternatively, after hybridization of the target nucleic acid molecule to the oligonucleotide probes, the hybridized target nucleic acid molecule is coated with a conductive material, such as a metal, as described in U.S. Patent Applications Serial Nos. 60/095,096 or 60/099,506, which are hereby incorporated by reference in their entirety. Examples of conductive material include silver and gold. The coated nucleic acid molecule can then conduct electricity across the gap between the pair of probes, thus producing a detectable signal indicative of the presence of a target nucleic acid molecule. Thus, the present invention relates to a method for detecting a target nucleic acid molecule in a sample. The method first involves providing an apparatus which includes first and second electrical conductors each having detection sites located less than 250 microns apart but not in contact with one another. The first electrical conductor is made of a first type of conductive material and the second electrical conductor is made of a second type of conductive material which is different than the first type of conductive material. The apparatus also includes a first set of oligonucleotide probes attached to the detection sites of the first electrical conductors with an attachment chemistry which binds the first set of oligonucleotide probes to the first electrical conductor but not to the second electrical conductor. Finally, the apparatus includes a second set of oligonucleotide probes attached to the detection sites of the second electrical conductors and spaced apart from the first set of oligonucleotide probes by a gap. Next, the probes are contacted with a sample potentially containing a target nucleic acid molecule under conditions effective to permit any of the target nucleic acid molecule in the sample to hybridize to both of the spaced apart oligonucleotide probes to bridge the gap and electrically couple the pair of oligonucleotide probes with the hybridized target nucleic acid molecule, if any. A conductive material is then applied over the electrically coupled pair of oligonucleotide probes and the hybridized target nucleic acid molecule. Finally, it is determined if an electrical current can be carried between the probes, where the electrical current between the probes indicates the presence of the target nucleic acid molecule in the sample which has sequences complementary to the probes.

For instance, the sodium counter ions to DNA phosphate groups can be replaced with silver ions by flooding the sample area with silver nitrate solution. After washing away excess silver nitrate, bathing the area with a photographic developer such as hydroquinone reduces the silver ions to metallic silver, which is electrically conductive. Braun et al. demonstrated that silver could be deposited along a DNA molecule (Braun et al., “DNA-Templated Assembly and Electrode Attachment of a Conducting Silver Wire,” Nature, 391:775-778 (1998), which is hereby incorporated in its entirety). A three-step process is used. First, silver is selectively localized to the DNA molecule through a Ag+/Na+ ion-exchange (Barton, Bioinorganic Chemistry, eds. Bertini, et al., ch. 8, University Science Books, Mill Valley, (1994), which is hereby incorporated by reference in its entirety) and complexes are formed between the silver and the DNA bases (Spiro, ed., Nucleic Acid-Metal Ion Interactions, Wiley Interscience, New York (1980); Marzeilli, et al., J. Am. Chem. Soc., 99:2797 (1977); Eichorn, ed. Inorganic Biochemistry, Vol. 2, ch 33-34, Elsevier, Amsterdam, (1973), which are hereby incorporated by reference in their entirety). The ion-exchange process may be monitored by following the quenching of the fluorescence signal of the labeled DNA. The silver ion-exchanged DNA is then reduced to form aggregates with bound to the DNA skeleton. The silver aggregates are further developed using standard procedures, such as those used in photographic chemistry (Holgate, et al., J. Histochem. Cytochem., 31:938 (1983); Birell, et al., J. Histochem. Cytochem., 34:339 (1986), which are hereby incorporated by reference in their entirety).

The target nucleic acid molecule, whose sequence is to be determined, is usually isolated from a tissue sample. If the target nucleic acid molecule is genomic, the sample may be from any tissue (except exclusively red blood cells). For example, saliva, whole blood, peripheral blood lymphocytes, or PBMC, skin, hair or semen are convenient sources of clinical samples. These sources are also suitable if the target is RNA. Blood and other body fluids are also a convenient source for isolating viral nucleic acids. If the target is mRNA, the sample is obtained from a tissue in which the mRNA is expressed. If the polynucleotide in the sample is RNA, it may be reverse transcribed to DNA, but in this method need not be converted to DNA.

For those embodiments where whole cells, viruses or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like.

Liberation of nucleic acids from the sample cells or viruses, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within the extraction chamber, a separate accessible chamber or externally introduced.

Alternatively, physical methods may be used to extract the nucleic acids and denature DNA binding proteins. U.S. Pat. No. 5,304,487, which is hereby incorporated by reference in its entirety, discusses the use of physical protrusions within microchannels or sharp edged particles within a chamber or channel to pierce cell membranes and extract their contents. More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, or forcing cells through microgeometry apertures, thereby subjecting the cells to high shear stress resulting in rupture.

Following extraction, it will often be desirable to separate the nucleic acids from other elements of the crude extract, e.g., denatured proteins, cell membrane particles, and the like. Removal of particulate matter is generally accomplished by filtration, flocculation, or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample. Suitable solid supports for nucleic acid binding include, e.g., diatomaceous earth, silica, or the like. Suitable gel exclusion media is also well known in the art and is commercially available from, e.g., Pharmacia and Sigma Chemical. This isolation and/or gel filtration/desalting may be carried out in an additional chamber, or alternatively, the particular chromatographic media may be incorporated in a channel or fluid passage leading to a subsequent reaction chamber.

Alternatively, the interior surfaces of one or more fluid passages or chambers may themselves be derivatized to provide functional groups appropriate for the desired purification, e.g., charged groups, affinity binding groups and the like.

The oligonucleotide probes of the present invention may be designed to specifically recognize a variation in the sequence at the end of the probe. After the target nucleic acid molecule binds to the probes, the target nucleic acid molecule is treated with nucleases to remove the ends of the molecule which do not bind to the probes. If the confronting ends of the two probes contain sequences complementary to the target nucleic acid molecule, treatment with ligase will join the confronting ends of the two probes. The test chamber can then be heated up to denature non-ligated target nucleic acid molecule from the probes. Detection of the specific target nucleic acid molecule can then be carried out.

In a preferred embodiment of the invention, ligation methods may be used to specifically identify single base differences in sequences. Previously, methods of identifying known target sequences by probe ligation methods have been reported (U.S. Pat. No. 4,883,750 to N. M. Whiteley et al.; Wu et al., Genomics, 4:560 (1989); Landegren et al., Science, 241:1077 (1988); and Winn-Deen et al., Clin. Chem., 37:1522 (1991), which are hereby incorporated by reference in their entirety). In one approach, known as oligonucleotide ligation assay (“OLA”), two probes or probe elements which span a target region of interest are hybridized to the target region. Where the probe elements basepair with adjacent target bases, the confronting ends of the probe elements can be joined by ligation, e.g., by treatment with ligase. The ligated probe element is then assayed, evidencing the presence of the target sequence.

Homologous nucleotide sequences can be detected by selectively hybridizing to each other. Selectively hybridizing is used herein to mean hybridization of DNA or RNA probes from one sequence to the “homologous” sequence under stringent or non-stringent conditions (Ausubel et al., eds., Current Protocols in Molecular Biology, Vol. I: 2.10.3, Greene Publishing Associates, Inc. and John Wiley & Sons, Inc., New York (1989), which is hereby incorporated by reference in its entirety). Hybridization and wash conditions are also exemplified in Sambrook et al., Molecular Cloning: A Laboratory Manual, Second Edition, Cold Spring Harbor, N.Y. (1989), which is hereby incorporated by reference in its entirety.

A variety of hybridization buffers are useful for the hybridization assays of the invention. Addition of small amounts of ionic detergents (such as N-lauroyl-sarkosine) are useful. LiCl is preferred to NaCl. Additional examples of hybridization conditions are provided in several sources, including: Sambrook et al., Molecular Cloning: A Laboratory Manual, 2nd Ed., Cold Spring Harbor, N.Y. (1989); Berger et al., “Guide to Molecular Cloning Techniques,” Methods in Enzymology, Volume 152, Academic Press, Inc., San Diego, Calif. (1987); and Young et al., Proc. Natl. Acad. Sci. USA, 80:1194 (1983), which are hereby incorporated by reference in their entirety. In addition to aqueous buffers, non-aqueous buffers may also be used. In particular, non-aqueous buffers which facilitate hybridization but have low electrical conductivity are preferred.

The hybridization mixture is placed in contact with the array and incubated. Contact can take place in any suitable container, for example, a dish or a cell specially designed to hold the probe array and to allow introduction of the fluid into and removal of it from the cell so as to contact the array. Generally, incubation will be at temperatures normally used for hybridization of nucleic acids, for example, between about 20° C. and about 75° C., e.g., about 25° C., about 30° C., about 35° C., about 40° C., about 45° C., about 50° C., about 55° C., about 60° C., or about 65° C. For probes longer than about 14 nucleotides, 37-45° C. is preferred. For shorter probes, 55-65° C. is preferred. More specific hybridization conditions can be calculated using formulae for determining the melting point of the hybridized region. Preferably, hybridization is carried out at a temperature at or between ten degrees below the melting temperature and the melting temperature. More preferred, the hybridization is carried out at a temperature at or between five degrees below the melting temperature and the melting temperature. The target is incubated with the probe array for a time sufficient to allow the desired level of hybridization between the target and any complementary probes in the array. After incubation with the hybridization mixture, the array usually is washed with the hybridization buffer, which also can include the hybridization optimizing agent. These agents can be included in the same range of amounts as for the hybridization step, or they can be eliminated altogether. Then, the array can be examined to identify the probes to which the target has hybridized.

The number of probes may be increased in order to determine concentrations of the target nucleic acid molecule. If a plurality of each pair of oligonucleotide probes is provided, the method of the present invention can be used to identify the number of pairs of identical oligonucleotide probes between which electrical current passes to quantify the amount of the target nucleic acid molecule present in the sample. For example, several thousand repeated probes may be produced in the detection apparatus. The circuit would be able to count the number of occupied sites. Calculations could be done by the unit to determine the concentration of the target nucleic acid molecule.

The method of the present invention can be used for numerous applications, such as detection of pathogens or viruses. For example, samples may be isolated from drinking water or food and rapidly screened for infectious organisms, using probes that are complementary to the genetic material of a pathogenic bacteria. In recent times, there have been several large recalls of tainted meat products. The method of the present invention can be used for the in-process detection of pathogens in foods and the subsequent disposal of the contaminated materials. This could significantly improve food safety, prevent food borne illnesses and death, and avoid costly recalls. Detection devices with oligonucleotide probes that are complementary to the genetic material of common food borne pathogens, such as Salmonella and E. coli., could be designed for use within the food industry.

In yet another embodiment, the method of the present invention can be used for real time detection of biowarfare agents, by using probes that are complementary to the genetic material of a biowarfare agent. With the recent concerns of the use of biological weapons in a theater of war and in terrorist attacks, the device could be configured into a personal sensor for the combat soldier or into a remote sensor for advanced warnings of a biological threat. The devices which can be used to specifically identity of the agent, can be coupled with a modem to send the information to another location. Mobile devices may also include a global positioning system to provide both location and pathogen information.

In yet another embodiment, the present invention may be used to identify an individual, by using probes that are complementary to the genetic material of a human. A series of probes, of sufficient number to distinguish individuals with a high degree of reliability, are placed within the device. Various polymorphism sites are used. Preferentially, the device can determine the identity to a specificity of greater than one in 1 million, more preferred is a specificity of greater than one in one billion, even more preferred is a specificity of greater than one in ten billion. The present invention may be used to screen for mutations or polymorphisms in samples isolated from patients.

This invention may also be used for nucleic acid sequencing using hybridization techniques. Such methods are described in U.S. Pat. No. 5,837,832, which is hereby incorporated by reference in its entirety.

### EXAMPLES

The following examples are provided to illustrate embodiments of the present invention but are by no means intended to limit its scope.

### Example 1

### Attaching Oligonucleotide Probes to Aluminum Electrical Conductors

A 1 cm square chip of silicon having a 300 nm layer of sputtered aluminum on its surface is submersed in a solution of 1000 microliters of 30% hydrogen peroxide mixed with 100 microliters of concentrated ammonium hydroxide and allowed to sit at room temperature of 20 minutes. The chip is then rinsed with pure water and allowed to air dry. The chip is then submersed into a solution of 1 μl N-[3-(trimethoxysilyl)-propyl]ethlenediamine, sold as product Z-6094 by the Dow Corning Company (Midland, Mich.), in 10 ml of toluene. After 15 minutes, the chip is rinsed in toluene and air-dried. Then, the chip is submerged in a solution of 0.03% N-succinimidy-(4-vinylsulfonyl)benzoate in 90:10 (100 mM sodium phosphate buffer, pH=8: dimethylsulfoxide), and incubated for 30 minutes. The chip is then washed with dimethylsulfoxide, water, and ethanol and allowed to air dry. A solution (5 picomoles in 50 microliters) of P-32 radioactively labeled oligonucleotide in 100 mM phosphate buffer, pH=7, was then placed on the chip and allowed to sit for 30 minutes. The chip was then washed in 100 mM phosphate buffer (pH=7) containing 0.1% sodium dodecylsulfate by agitating the chip for about 1 minute. The chip was rinsed in water and then placed in a scintillation vial with 5 ml of scintillation fluid. The scintillation counter recorded 25,000 CPM, indicating there was, on average, one oligonucleotide molecule for each 900 square nanometers on the chip. The radioactive signal was not removed by continued washing in SDS phosphate buffer.

### Example 2

### Attaching Oligonucleotide Probes to Gold Electrical Conductors

A 1 cm square chip of silicon having a 1 nm layer of sputter titanium on its surface, and over the titanium, a 100 nm layer of sputtered gold is submersed in a solution of 1000 microliters of 30% hydrogen peroxide mixed with 100 microliters of glacial acetic acid and allowed to sit at room temperature for 20 minutes. The chip is then rinsed with pure water and allowed to air dry. A solution (5 picomoles in 50 microliters) of P-32 radioactively labeled oligonucleotide in 100 mM phosphate buffer, pH=7, was then placed on the chip and allowed to sit for 30 minutes. The chip was then washed in 100 mM phosphate buffer (pH=7) containing 0.1% sodium dodecylsulfate by agitating the chip for about 1 minute. The chip was rinsed in water and then placed in a scintillation vial with 5 ml of scintillation fluid. The scintillation counter recorded 128,000 CPM, indicating there was, on average, one oligonucleotide molecule for each 84 square nanometers on the chip. The radioactive signal was not removed by continued washing in SDS phosphate buffer.

### Example 3

### Attaching Oligonucleotide Probes to Gold Electrical Conductors

A 1 cm square chip of silicon having a 1 nm layer of sputtered titanium on its surface, and over the titanium, a 100 nm layer of sputtered gold is submersed in a solution of 1000 microliters of 30% hydrogen peroxide mixed with 100 microliters of glacial acetic acid and allowed to sit at room temperature for 20 minutes. The chip is then rinsed with pure water and allowed to air dry. A solution (5 picomoles in 50 microliters) of P-32 radioactively labeled oligonucleotide in 95:5 dimethylsulfoxide:water was then placed on the chip and allowed to sit for 5 minutes. The chip was then washed in 100 mM phosphate buffer (pH=7) containing 0.1% sodium dodecylsulfate by agitating the chip for about 1 minute. The chip was rinsed in water and then placed in a scintillation vial with 5 ml of scintillation fluid. The counts recorded from the scintillation counter were comparable to those obtained in Example 2. The radioactive signal was not removed by continued washing in SDS phosphate buffer.

### Example 4

### Attaching Oligonucleotide Probes to Gold Electrical Conductors

A 1 cm square chip of silicon having a 1 nm layer of sputter titanium on its surface, and over the titanium, a 100 nm layer of sputtered gold is submersed in a solution of 1000 microliters of 30% hydrogen peroxide mixed with 100 microliters of glacial acetic acid and allowed to sit at room temperature for 20 minutes. The chip is then rinsed with pure water and allowed to air dry. A solution (5 picomoles in 50 microliters) of P-32 radioactively labeled oligonucleotide in 95:5 dimethylsulfoxide:water was then placed on the chip and allowed to sit for 5 minutes. Then, 10 microliters of a solution of 0.1% dodecanethiol in dimethylsulfoxide was added to the chip and allowed to stand for 1 minute. The chip was then washed in 100 mM phosphate buffer (pH=7) containing 0.1% sodium dodecylsulfate by agitating the chip for about 1 minute. The chip was rinsed in water and then placed in a scintillation vial with 5 ml of scintillation fluid. The counts recorded from the scintillation counter were comparable to those obtained in Example 3. The radioactive signal was not removed by continued washing in SDS phosphate buffer. The dodecanethiol evidently occupies and blocks any active sites on the gold surface and thus prevents further oligonucleotide binding, since further applications of radioactive probe solution did not produce further increases in bound radioactive scintillation counts.

### Example 5

### Attaching PNA Probes to Gold Electrical Conductors

A 1 cm square chip of silicon having a 30 nm layer of sputtered chromium on its surface, and, over the chromium, a 100 nm layer of sputtered gold is submersed in a solution of 1000 microliters of 30% hydrogen peroxide mixed with 100 microliters of concentrated ammonium hydroxide and allowed to sit at room temperature for 20 minutes. The chip is then rinsed with pure water and allowed to air dry. A solution (2 picomoles in 50 microliters) of PNA probe terminated with a cysteine amino acid (18-mer, made by the Applied Biosystems Company, Framingham, Mass.) in 100 mM phosphate buffer, pH=7.8, with 0.1% SDS added, was then placed on the chip and allowed to sit for about 15 minutes. The chip was then washed in washing buffer for about 1 minute, rinsed in water and then covered with a solution of 5 picomoles of P-32 radioactively labeled DNA containing a complementary sequence to the PNA probe in 50 microliters of 100 mM phosphate buffer, pH=7.8, with 0.1% SDS added. The solution was applied at 70° C., with the chip at 55° C. The chip was held at 55° C. for about 5 minutes, and then allowed to gradually cool to room temperature over a period of about 20 minutes. The chip was then washed for about 1 minute in washing buffer, rinsed with water and placed in a scintillation vial with 5 ml of scintillation fluid. The counts recorded on the scintillation counter were comparable to those obtained in Example 2. The radioactive signal was not removed by continued washing with the washing buffer, showing that the PNA probe was bound to the gold surface.

### Example 6

### Attaching Oligonucleotide Probes to Indium Tin Oxide (ITO) Electrical Conductors

A 1 cm square of polyethyleneterphthalate support having a 500 nm layer of conductive ITO on its surface is submersed in a solution of 1000 microliters of 30% hydrogen peroxide mixed with 100 microliters of concentrated ammonium hydroxide and allowed to sit at room temperature for 20 minutes. The chip is then rinsed with pure water and allowed to air dry. The chip is then submersed into a solution of 1 microliter of N-[3-(trimethoxysilyl)-propyl]ethlenediamine, sold as Z6094 by the Dow Corning Company, in 10 ml of toluene. After 15 minutes, the chip is rinsed in toluene and air-dried. Then, the chip is submersed in a solution of 0.03% N-succinimidyl-(4-vinylsulfonyl)benzoate in 90:10 (100 mM sodium phosphate buffer, pH=8: dimethylsulfoxide), and incubated for 30 minutes. The chip is then washed with dimethylsulfoxide, water, and ethanol, and allowed to air dry. A solution (5 picomoles in 50 microliters) of P-32 radioactively labeled oligonucleotide (36-mer, made by the Sigma Genesis Company, The Woodlands, Tex.) in 100 mM phosphate buffer, pH=7, was then placed on the chip and allowed to sit for 30 minutes. The chip was then washed in 100 mM phosphate buffer (pH=7) containing 0.1% sodium dodecylsulfate (SDS), hereafter termed the “washing buffer”, by agitating the chip for about 1 minute. The chip was then rinsed in water and then placed in a scintillation vial with 5 ml of scintillation fluid. The counts recorded on the scintillation counter were comparable to those obtained in Example 1. The radioactive signal was not removed by continued washing with the washing buffer, showing the PNA probe was bound to the gold surface.

### Example 7

### Attaching Oligonucleotide Probes to Amorphous Silicon Electrical Conductors

A 1 cm square of silicone support having a 500 nm layer of conductive amorphous silicon on its surface is submersed in a solution of 1000 microliters of 30% hydrogen peroxide mixed with 100 microliters of concentrated ammonium hydroxide and allowed to sit at room temperature for 20 minutes. The chip is then rinsed with pure water and allowed to air dry. The chip is then submersed into a solution of 1 microliter of N-[3-(trimethoxysilyl)-propyl]ethlenediamine, sold as Z6094 by the Dow Corning Company, in 10 ml of toluene. After 15 minutes, the chip is rinsed in toluene and air-dried. Then, the chip is submersed in a solution of 0.03% N-succinimidyl-(4vinylsulfonyl)benzoate in 90:10 (100 mM sodium phosphate buffer, pH=8: dimethylsulfoxide), and incubated for 30 minutes. The chip is then washed with dimethylsulfoxide, water, and ethanol, and allowed to air dry. A solution (5 picomoles in 50 microliters) of P-32 radioactively labeled oligonucleotide (36-mer, made by the Sigma Genesis Company, The Woodlands, Tex.) in 100 mM phosphate buffer, pH=7, was then placed on the chip and allowed to sit for 30 minutes. The chip was then washed in 100 mM phosphate buffer (pH=7) containing 0.1% sodium dodecylsulfate (SDS), hereafter termed the “washing buffer”, by agitating the chip for about 1 minute. The chip was then rinsed in water and then placed in a scintillation vial with 5 ml of scintillation fluid. The counts recorded on the scintillation counter were comparable to those obtained in Example 1. The radioactive signal was not removed by continued washing with the washing buffer, showing the PNA probe was bound to the gold surface.

Although the invention has been described in detail for the purpose of illustration, it is understood that such detail is solely for that purpose, and variations can be made therein by those skilled in the art without departing from the spirit and scope of the invention that is defined by the following claims.

What is claimed:

1. A method of attaching nucleic acid molecules to electrically conductive surfaces, said method comprising:
providing first and second electrical conductors, located near but not in contact with one another, wherein the first electrical conductor is made of a first type of conductive material and the second electrical conductor is made of a second type of conductive material which is different than the first type of conductive material;
attaching a first set of oligonucleotide probes to the first electrical conductor with an attachment chemistry which binds the first set of oligonucleotide probes to the first electrical conductor but not to the second electrical conductor; and
attaching a second set of oligonucleotide probes to the second electrical conductor.

2. A method according to claim 1 further comprising:
attaching blocking molecules to the first electrical conductor at all sites not occupied by the first set of oligonucleotide probes after said attaching a first set of oligonucleotide probes and before said attaching a second set of oligonucleotide probes.

3. A method according to claim 2 further comprising:
functionalizing a surface of the second electrical conductor, after said attaching blocking molecules and before said attaching a second set of oligonucleotide probes to permit the second set of oligonucleotide probes to be attached to the second electrical conductor.

4. A method according to claim 3, wherein the surface of the second electrical conductor is functionalized with hydroxyl groups.

5. A method according to claim 2, wherein the first type of conductive material is gold, the second type of conductive material is aluminum, the attachment chemistry for the first electrical conductor is a mercapto group, and the blocking molecules have thiol groups which are attached to the first electrical conductor.

6. A method according to claim 1, wherein the second set of oligonucleotide probes is attached to the second electrical conductor by silanizing a surface of the second electrical conductor and linking the silanized surface of the second electrical conductor to the second set of oligonucleotide probes with a siloxane group.

7. A method according to claim 1, wherein the first and second electrical conductors are fixed on a substrate.

8. A method according to claim 7, wherein the substrate is selected from the group consisting of glass, quartz, silicon, and polymeric material.

9. A method of attaching nucleic acid molecules to electrically conductive surfaces, said method comprising:
providing first and second electrical conductors located near, but not in contact with one another, wherein the second electrical conductor is covered with a masking agent;
attaching a first set of oligonucleotide probes to the first electrical conductor with an attachment chemistry which binds the first set of oligonucleotide probes to the first electrical conductor;
removing the masking agent from the second electrical conductor; and
attaching a second set of oligonucleotide probes to the second electrical conductor with an attachment chemistry which binds the second set of oligonucleotide probes to the second electrical conductor.

10. A method according to claim 9 further comprising:
attaching blocking molecules to the first or second electrical conductors at all sites not occupied by the first or second set of oligonucleotide probes after said attaching a first set of oligonucleotide probes or said attaching a second set of oligonucleotide probes.

11. A method according to claim 9, wherein the first and second electrical conductors are covered with a masking agent, said method further comprising:
removing the masking agent from the first electrical conductor but not from the second electrical conductor prior to said attaching a first set of oligonucleotide probes to the first electrical conductors.

12. A method according to claim 11, wherein the masking agent is photoresist and said removing the masking agent from the first or second electrical conductor is carried out by a process comprising:
exposing the photoresist at a location corresponding to the first or second electrical conductor with radiation; and
removing the exposed photoresist.

13. A method according to claim 9, wherein the first and second conductors are made of the same type of material.

14. A method according to claim 10, wherein the first and second electrical conductors are made of gold, the attachment chemistry for the first and second electrical conductors is a mercapto group, and the blocking molecules have thiol groups attached to the first and second electrical conductors.

15. A method according to claim 9, wherein the first and second electrical conductors are fixed on a substrate.

16. A method according to claim 15, wherein the substrate is selected from the group consisting of glass, quartz, silicon, and polymeric material.

17. A method of attaching multiple oligonucleotide probe molecules to electrically conductive surfaces, said method comprising:
providing first and second electrical conductors, located near but not in contact with one another;
attaching metal particles to the first electrical conductor by silanizing a surface of the first electrical conductor and linking the silanized surface to the metal particles with a siloxane group; and
attaching multiple oligonucleotide probe molecules to said metal particles attached to the first electrical conductor.

18. A method according to claim 17 further comprising:
attaching metal particles to the second electrical conductor by silanizing a surface of the second electrical conductor and linking the silanized surface to the metal particles with a siloxane group; and
attaching multiple oligonucleotide probe molecules to said metal particles attached to the second electrical conductor.

19. A method according to claim 17, wherein the first electrical conductor is made of aluminum and the metal particles are made of gold.

20. A method of attaching nucleic acid molecules to electrically conductive surfaces, said method comprising:
providing first and second electrical conductors located near, but not in contact with one another, wherein a voltage source is connected to said electrical conductors; and
attracting a first set of oligonucleotide probes toward the first electrical conductor by making the first electrical conductor more positively charged relative to the second electrical conductor, wherein the first set of oligonucleotide probes chemically binds to the first electrical conductor.

21. A method according to claim 20 further comprising:
attracting a second set of oligonucleotide probes toward the second electrical conductor by making the second electrical conductor more positively charged relative to the first electrical conductor, wherein the second set of oligonucleotide probes chemically binds to the second electrical conductor.

22. A method according to claim 21, wherein during said attracting a first set of oligonucleotide probes, the first electrical conductor is positively charged and the second electrical conductor is negatively charged, and during said attracting a second set of oligonucleotide probes, the second electrical conductor is positively charged and the first electrical conductor is negatively charged.

23. A method according to claim 20 further comprising:
attaching blocking molecules to the first electrical conductor at all sites not occupied by the first set of oligonucleotide probes after said first set of oligonucleotide probes binds to the first electrical conductor.

24. A method according to claim 20 further comprising:
electroplating the first electrical conductor with a specific metal prior to said attracting a first set of oligonucleotide probes.

25. A method according to claim 21 further comprising:
electroplating the second electrical conductor with a specific metal prior to said attracting a second set of oligonucleotide probes.

26. A method according to claim 20, wherein the first and second conductors are made of the same type of material.

27. An apparatus for detecting a target nucleic acid molecule in a sample, said apparatus comprising:
first and second electrical conductors, each having detection sites located less than 250 microns apart but not in contact with one another, wherein the first electrical conductor is made of a first type of conductive material and the second electrical conductor is made of a second type of conductive material which is different than the first type of conductive material;
a first set of oligonucleotide probes attached to the detection sites of the first electrical conductors with an attachment chemistry which binds the first set of oligonucleotide probes to the first electrical conductor but not to the second electrical conductor; and
a second set of oligonucleotide probes attached to the detection sites of the second electrical conductors.

28. An apparatus according to claim 27, wherein the detection sites are located less than 100 microns apart.

29. An apparatus according to claim 27, wherein the detection sites are located less than 10 microns apart.

30. An apparatus according to claim 27, wherein blocking molecules are attached to the first electrical conductor at all sites not occupied by the first set of oligonucleotide probes.

31. An apparatus according to claim 30, wherein the first type of conductor material is gold, the second type of conductor material is aluminum, the attachment chemistry for the first type of conductor material is a mercapto group, and the blocking molecules have thiol groups attached to the first electrical conductor.

32. An apparatus according to claim 27, wherein the second set of oligonucleotide probes is attached to the second electrical conductor by silanizing a surface of the second electrical conductor and linking the silanized surface of the second electrical conductor to the second set of oligonucleotide probes with a siloxane group.

33. An apparatus according to claim 27, wherein the first and second electrical conductors are fixed on a substrate.

34. An apparatus according to claim 33, wherein the substrate is selected from the group consisting of glass, quartz, silicon, and polymeric material.

35. A method for detecting a target nucleic acid molecule in a sample comprising:
providing an apparatus comprising:
first and second electrical conductors, each having detection sites located less than 250 microns apart but not in contact with one another, wherein the first electrical conductor is made of a first type of conductive material and the second electrical conductor is made of a second type of conductive material which is different than the first type of conductive material;
a first set of oligonucleotide probes attached to the detection sites of the first electrical conductors with an attachment chemistry which binds the first set of oligonucleotide probes to the first electrical conductor but not to the second electrical conductor; and
a second set of oligonucleotide probes attached to the detection sites of the second electrical conductors and spaced apart from the first set of oligonucleotide probes by a gap;
contacting the probes with a sample potentially containing a target nucleic acid molecule under conditions effective to permit any of the target nucleic acid molecule in the sample to hybridize to both of the spaced apart oligonucleotide probes, thereby bridging the gap and electrically coupling the pair of oligonucleotide probes with the hybridized target nucleic acid molecule, if any;
filling the electrically coupled pair of oligonucleotide probes and the hybridized target nucleic acid molecule with a filling nucleic acid sequence, wherein the filling nucleic acid sequence is complementary to the target nucleic acid molecule and extends between the pair of oligonucleotide probes; and
determining if an electrical current can be carried between the probes, said electrical current between the probes indicating the presence of the target nucleic acid molecule in the sample which has sequences complementary to the probes.

36. A method according to claim 35, wherein the target nucleic acid molecule is DNA.

37. A method according to claim 35, wherein the target nucleic acid molecule is RNA.

38. A method according to claim 35 further comprising;
coating the oligonucleotide probes as well as any target nucleic acid molecule with a conductive material.

39. A method according to claim 38, wherein the conductive material is silver.

40. A method according to claim 38, wherein the conductive material is gold.

41. A method according to claim 35 further comprising:
contacting the target nucleic acid molecule with nucleases after binding with the probes.

42. A method according to claim 35, wherein the first and second oligonucleotide probes abut one another at a junction when hybridized to the target nucleic acid molecule, said method further comprising:
contacting the target nucleic acid molecule with ligase after said filling; and
heating the apparatus to a temperature high enough to denature the target nucleic acid molecule from the probes.

43. A method according to claim 35, wherein the probes are complementary to the genetic material of a pathogenic bacteria.

44. A method according to claim 43, wherein the pathogenic bacteria is a biowarfare agent.

45. A method according to claim 43, wherein the pathogenic bacteria is a food borne pathogen.

46. A method according to claim 35, wherein the probes are complementary to the genetic material of a virus.

47. A method according to claim 35, wherein the probes are complementary to the genetic material of a human.

48. A method according to claim 35, wherein the probes have a sequence which is complementary to a sequence containing a polymorphism.

49. A method according to claim 35, wherein a plurality of each pair of oligonucleotide probes is provided, said method further comprising:
identifying the number of pairs of identical oligonucleotide probes between which electrical current passes to quantify the amount of the target nucleic acid molecule present in the sample.

50. A method according to claim 35, wherein the pair of oligonucleotide probes are configured to hybridize to the target nucleic acid molecule at a temperature of 20-75° C.

51. A method according to claim 35 further comprising:
removing any portion of the target nucleic acid molecule which does not hybridize to the pair of oligonucleotide probes with a nuclease after said contacting.

52. A method according to claim 35, wherein the first and second electrical conductors are fixed on a substrate.

53. A method according to claim 52, wherein the substrate is selected from the group consisting of glass, quartz, silicon, and polymeric material.

54. A method according to claim 35, wherein the sample is saliva, whole blood, peripheral blood lymphocytes, skin, hair, or semen.

55. A method according to claim 35, wherein said method is used to detect infectious agents.

56. A method according to claim 35, wherein said method is used for nucleic acid sequencing.

57. A method according to claim 35, wherein the detection sites are located less than 100 microns apart.

58. A method according to claim 35, wherein the detection sites are located less than 10 microns apart.

59. A method according to claim 35, wherein blocking molecules are attached to the first electrical conductors at all sites not occupied by the first set of oligonucleotide probes.

60. A method according to claim 59, wherein the first type of conductor is gold, the second type of conductor is aluminum, the attachment chemistry for the first type of conductor is a mercapto group, and the blocking molecules have thiol groups attached to the first type of conductor.

61. A method according to claim 35, wherein the second set of oligonucleotide probes is attached to the second type of conductor by silanizing the surfaces of the second conductors and linking the silanized surfaces to the second set of oligonucleotide probes with a siloxane group.

62. A method for detecting a target nucleic acid molecule in a sample comprising:
providing an apparatus comprising:
first and second electrical conductors, each having detection sites located less than 250 microns apart but not in contact with one another, wherein the first electrical conductor is made of a first type of conductive material and the second electrical conductor is made of a second type of conductive material which is different than the first type of conductive material;
a first set of oligonucleotide probes attached to the detection sites of the first electrical conductors with an attachment chemistry which binds the first set of oligonucleotide probes to the first electrical conductor but not to the second electrical conductor; and
a second set of oligonucleotide probes attached to the detection sites of the second electrical conductors and spaced apart from the first set of oligonucleotide probes by a gap;
contacting the probes with a sample potentially containing a target nucleic acid molecule under conditions effective to permit any of the target nucleic acid molecule in the sample to hybridize to both of the spaced apart oligonucleotide probes, thereby bridging the gap and electrically coupling the pair of oligonucleotide probes with the hybridized target nucleic acid molecule, if any;
applying a conductive material over the electrically coupled pair of oligonucleotide probes and the hybridized target nucleic acid molecule; and
determining if an electrical current can be carried between the probes, said electrical current between the probes indicating the presence of the target nucleic acid molecule in the sample which has sequences complementary to the probes.

63. A method according to claim 62, wherein the target nucleic acid molecule is DNA.

64. A method according to claim 62, wherein the target nucleic acid molecule is RNA.

65. A method according to claim 62, wherein the conductive material is silver.

66. A method according to claim 62, wherein the conductive material is gold.

67. A method according to claim 62 further comprising:
contacting the target nucleic acid molecule with nucleases after binding with the probes.

68. A method according to claim 62, wherein the first and second oligonucleotide probes abut one another at a junction when hybridized to the target nucleic acid molecule, said method further comprising:
contacting the target nucleic acid molecule with ligase after said filling; and
heating the apparatus to a temperature high enough to denature the target nucleic acid molecule from the probes.

69. A method according to claim 62, wherein the probes are complementary to the genetic material of a pathogenic bacteria.

70. A method according to claim 69, wherein the pathogenic bacteria is a biowarfare agent.

71. A method according to claim 69, wherein the pathogenic bacteria is a food borne pathogen.

72. A method according to claim 62, wherein the probes are complementary to the genetic material of a virus.

73. A method according to claim 62, wherein the probes are complementary to the genetic material of a human.

74. A method according to claim 62, wherein the probes have a sequence which is complementary to a sequence containing a polymorphism.

75. A method according to claim 62, wherein a plurality of each pair of oligonucleotide probes is provided, said method further comprising:
identifying the number of pairs of identical oligonucleotide probes between which electrical current passes to quantify the amount of the target nucleic acid molecule present in the sample.

76. A method according to claim 62, wherein the pair of oligonucleotide probes are configured to hybridize to the target nucleic acid molecule at a temperature of 20-75° C.

77. A method according to claim 62 further comprising:
removing any portion of the target nucleic acid molecule which does not hybridize to the pair of oligonucleotide probes with a nuclease after said contacting.

78. A method according to claim 62, wherein the first and second electrical conductors are fixed on a substrate.

79. A method according to claim 78, wherein the substrate is selected from the group consisting of glass, quartz, silicon, and polymeric material.

80. A method according to claim 62, wherein the sample is saliva, whole blood, peripheral blood lymphocytes, skin, hair, or semen.

81. A method according to claim 62, wherein said method is used to detect infectious agents.

82. A method according to claim 62, wherein said method is used for nucleic acid sequencing.

83. A method according to claim 62, wherein the detection sites are located less than 100 microns apart.

84. A method according to claim 62, wherein the detection sites are located less than 10 microns apart.

85. A method according to claim 62, wherein blocking molecules are attached to the first electrical conductors at all sites not occupied by the first set of oligonucleotide probes.

86. A method according to claim 85, wherein the first type of conductor is gold, the second type of conductor is aluminum, the attachment chemistry for the first type of conductor is a mercapto group, and the blocking molecules have thiol groups attached to the first type of conductor.

87. A method according to claim 62 wherein the second set of oligonucleotide probes is attached to the second type of conductor by silanizing the surfaces of the second conductors and linking the silanized surfaces to the second set of oligonucleotide probes with a siloxane group.
